SEQUENCE LISTING (7 sequences)

SEQ ID NO: 1 - DNA, 1422 bp, Sporobolus stapfianus
atgacgaagaccgtggttctgtacccgatccgcggcgccggccacctcacgccgatgattcagcttgccaggctcttcctccagcacggcggcttcaacgtcaccgtcgcaatcgggtcgtcccccgaggactccgacttctctgccctggtcgcgcgcgccgcggcggccaacccgtccgtcaccttccacatcctgcctcagccgtcctccacgccagacggttccaacaccgacgtcactcccaagcacaagcaccccgtggtccacttattcgacaccctcggcgccatgaacgcgccgctccgcgacttcctccgctcgctgccagccgtcgacgcgctcgtcgtggacatgttctgctacgacgcgctcgacgtcgcggccgagctcgagctgcccgcctacttcttgtacgcgtcgggcgccggcgacctcgccgtcttcctcaaccttcctagcgctcgagccggcatgacaacgagtttcgctgagctcggcgacactctgctcaccctgccgggtgcgccgccgttcaaagcttcggatttgcccgcggacgctatcaacgacaacgaggtggccaggtgcacccggcgcatgttcgagcggatgccggagtcccacgggattctcgtcaacagcttcgaggccctagagactcgtgcggtgcgcgctctgcgcgacgggctgtgcgttcccgaccgcgccacgccgccaatctactgcatcggacctttggtgtctggaggcggcggggaaaaggagcacgagtgcctccggtggctggacgcgcagccggacaacagcgtcgtgttcctctgcttcgggagcatgggaaccttctcgaagaagcagctccatgacatcgccgtcggcctggagaagtcagagcagaggtttctctgggtcgtgcggagcccacgcagcgacgaccacaagtttggtgagccacgtccggagctagacctggatgcgttcctgcgcgacgggttcctggagaggaccaaggagagaggactggtgctcaagtcgtgggcaccgcaggtggacgtgctccaccaccgtgcgaccggcgcgttcgtaacacactgtgggtggaactcgacgctggagggcatcatggcggggataccgctactgtgttggccattgtacgcggaacagaggatgaacaaggtgttcatcgtggacgagttgaagcttggggtcgagatgagaggctacaaccaggaggtagtgaaagccgaggaggtggagtcaaaggtcagatgggtgctggagtctgaggctggccaagcaataagagaacgggtgttggcgatgaaagacaaagcggccgaggcgctcaaggaaggaggcccatcccatgttgagtttgtcaagtttctgaaggacctggaggctgtcggtcgtccgtga

SEQ ID NO: 2 - DNA, 1740 bp, sporobolus stapfianus
ttgcgcacgtccccagcgcagcatccgaaggtctttcatctttggagtttagttcggcggcgagcgatgacgaagaccgtggttctgtacccgatccgcggcgccggccacctcacgccgatgattcagcttgccaggctcttcctccagcacggcggcttcaacgtcaccgtcgcaatcgggtcgtcccccgaggactccgacttctctgccctggtcgcgcgcgccgcggcggccaacccgtccgtcaccttccacatcctgcctcagccgtcctccacgccagacggttccaacaccgacgtcactcccaagcacaagcaccccgtggtccacttattcgacaccctcggcgccatgaacgcgccgctccgcgacttcctccgctcgctgccagccgtcgacgcgctcgtcgtggacatgttctgctacgacgcgctcgacgtcgcggccgagctcgagctgcccgcctacttcttgtacgcgtcgggcgccggcgacctcgccgtcttcctcaaccttcctagcgctcgagccggcatgacaacgagtttcgctgagctcggcgacactctgctcaccctgccgggtgcgccgccgttcaaagcttcggatttgcccgcggacgctatcaacgacaacgaggtggccaggtgcacccggcgcatgttcgagcggatgccggagtcccacgggattctcgtcaacagcttcgaggccctagagactcgtgcggtgcgcgctctgcgcgacgggctgtgcgttcccgaccgcgccacgccgccaatctactgcatcggacctttggtgtctggaggcggcggggaaaaggagcacgagtgcctccggtggctggacgcgcagccggacaacagcgtcgtgttcctctgcttcgggagcatgggaaccttctcgaagaagcagctccatgacatcgccgtcggcctggagaagtcagagcagaggtttctctgggtcgtgcggagcccacgcagcgacgaccacaagtttggtgagccacgtccggagctagacctggatgcgttcctgcgcgacgggttcctggagaggaccaaggagagaggactggtgctcaagtcgtgggcaccgcaggtggacgtgctccaccaccgtgcgaccggcgcgttcgtaacacactgtgggtggaactcgacgctggagggcatcatggcggggataccgctactgtgttggccattgtacgcggaacagaggatgaacaaggtgttcatcgtggacgagttgaagcttggggtcgagatgagaggctacaaccaggaggtagtgaaagccgaggaggtggagtcaaaggtcagatgggtgctggagtctgaggctggccaagcaataagagaacgggtgttggcgatgaaagacaaagcggccgaggcgctcaaggaaggaggcccatcccatgttgagtttgtcaagtttctgaaggacctggaggctgtcggtcgtccgtgaaagggcatgtttttttttattggaaagagcattgtttaacttgaatatgttggatttcaaaatgcacggatgccagttatacgtgtcatacatgtcttcagcttgcaaattattcaaacggattgagtacttagaaggtccttatttatgccacattggaacaacacaagatgttaattcatcaatcctgaagttagtcatggtcatttttgctatctaggtgttgtttgtggtggagttattgtgtgtatg

SEQ ID NO: 3 - protein, 473 aa, Sporobolus stapfianus
MTKTVVLYPIRGAGHLTPMIQLARLFLQHGGFNVTVAIGSSPEDSDFSALVARAAAANPSVTFHILPQPSSTPDGSNTDVTPKHKHPVVHLFDTLGAMNAPLRDFLRSLPAVDALVVDMFCYDALDVAAELELPAYFLYASGAGDLAVFLNLPSARAGMTTSFAELGDTLLTLPGAPPFKASDLPADAINDNEVARCTRRMFERMPESHGILVNSFEALETRAVRALRDGLCVPDRATPPIYCIGPLVSGGGGEKEHECLRWLDAQPDNSVVFLCFGSMGTFSKKQLHDIAVGLEKSEQRFLWVVRSPRSDDHKFGEPRPELDLDAFLRDGFLERTKERGLVLKSWAPQVDVLHHRATGAFVTHCGWNSTLEGIMAGIPLLCWPLYAEQRMNKVFIVDELKLGVEMRGYNQEVVKAEEVESKVRWVLESEAGQAIRERVLAMKDKAAEALKEGGPSHVEFVKFLKDLEAVGRP

SEQ ID NO: 4 - DNA, 846 bp, Sporobolus stapfianus
atgaccaaccgcatcttctttcccccatccatggcgacggcgaaccaggagtacataatccgattcgacggccagttcgaggacccctcgccgagctcctcgggcgtcgaaccacagccggtgcctgaggcggcgtcgttcccgtggaggacgatcagtcccgagcaggagcacgcggtcattgtgtccgccctgctccacgtcctctccggctacaccacgccgccgccccaggtcttcccggcgtcggcggctgcgcgagtggaggcgtgccggatgtgcgggatggagcggtgcctcggctgcgagttcttcgccgcggacgccggagtggtcgcgtcggatggatcagagaaggttcccgcggcagcagagacgacgacagctccggcggctgcaggagggcagaggcggcggcggaagaagaagaacaagtaccgcggcgtgaggcagcgtccgtgggggaagtgggcggcggagatccgcgacccgcgccgcgccgtgcgcaagtggctcggcacattcgacaccgccgaggaggccgccagggcgtacgaccgcgccgccgtcgagttccgcggcccgcgcgccaagctcaacttcccgttcccagagcaactggcagaccacgaagacgccagcgcggcagccaagtccgacacattgtcgccgtcgctgtgcagcggcagcgcggacgccgaggaccgtagcgttgggaggcaggagtggccgcaccagcacgggatggaagaaacaggggagcagctctgggacgacctccaggacctgatgaacctggacgaaggcgagatctggttcccgccaacctcgacaacttggaattga

SEQ ID NO: 5 - DNA, 842 bp, Sporobolus stapfianus
caagcttcttcttcttcacccactcactcgaggctcaactcagtcagggctcggtcatgaccaagaagctggatcccggcatggcaacgaagaataagcaaggtcgcgaggcgcagctcgacagtcaggggagacaggtttcgaactacgcggatggaggaactggaaggtcgacgatctctcaggagcaggagcacagcatcatggtcgcggcgctgcggcacgtcctgtccgggtacagcacgccgccgccggaggtggtcacggtggcggcgagcggcgaggcgtgcgggacctgcggcatcgacggctgcctcggctgcgacttcttcgcgggggaggagccccctcagctgctacgacagggactgagctatggcacaggcaccgcggcggtggcgacgagtaccgatgagaagaggaggaagcggcgcaggaagaggaacgcgttccgcggcgtgcggcagcggccatggggcaagtgggcggcggagatccgcgaccccaggaaggcggcgcgggtgtggctcggcacgttcgacaccgccgaggaggccgccagggcgtacgaccgcgccgcgctcgagttccgtggcgcgcgcgccaagctcaacttcccctaccaggaggagccggtcgtcctgctgggccatcacaatggcgattcggcggctgggacgacgctcacgccgccgtcgtcgtgcagcgtcgacgccgaggatcagagcgcggacggacttggggatgaactgtgggacggattgcaggacctgatgaagatggatgacgcggacttctggtcgttcgcgccgttctatggtgcagcatcgtctggtgtttga

SEQ ID NO: 6 - protein, 281 aa, Sporobolus stapfianus
MTNRIFFPPSMATANQEYIIRFDGQFEDPSPSSSGVEPQPVPEAASFPWRTISPEQEHAVIVSALLHVLSGYTTPPPQVFPASAAARVEACRMCGMERCLGCEFFAADAGVVASDGSEKVPAAAETTTAPAAAGGQRRRRKKKNKYRGVRQRPWGKWAAEIRDPRRAVRKWLGTFDTAEEAARAYDRAAVEFRGPRAKLNFPFPEQLADHEDASAAAKSDTLSPSLCSGSADAEDRSVGRQEWPHQHGMEETGEQLWDDLQDLMNLDEGEIWFPPTSTTWN

SEQ ID NO: 7 - protein, 263 aa, Sporobolus stapfianus
MTKKLDPGMATKNKQGREAQLDSQGRQVSNYADGGTGRSTISQEQEHSIMVAALRHVLSGYSTPPPEVVTVAASGEACGTCGIDGCLGCDFFAGEEPPQLLRQGLSYGTGTAAVATSTDEKRRKRRRKRNAFRGVRQRPWGKWAAEIRDPRKAARVWLGTFDTAEEAARAYDRAALEFRGARAKLNFPYQEEPVVLLGHHNGDSAAGTTLTPPSSCSVDAEDQSADGLGDELWDGLQDLMKMDDADFWSFAPFYGAASSGVGA

(19) United States
(12) Patent Application Publication
(10) Pub. No.: US 2017/0029839 A1
(43) Pub. Date: Feb. 2, 2017

(54) METHOD FOR IMPROVING CROP PRODUCTIVITY

(71) Applicant: Alan Donald NEALE, Parkdale, Victoria (AU)

(72) Inventors: Alan Donald NEALE, Parkdale, Victoria (AU); Cecilia Karstin Maria BLOMSTEDT, Collingwood, Victoria (AU); John Diarmuid HAMILL, Wheelers Hill, Victoria (AU); Donald Frederick GAFF, Donvale, Victoria (AU); Cara GRIFFITHS, Hoppers Crossing, Victoria (AU); Sharmin ISLAM, Indooroopilly, QLD (AU); Ngoc Tuan LE, Burwood, Victoria (AU)

(21) Appl. No.: 14/916,474

(22) PCT Filed: Sep. 1, 2014
(86) PCT No.: PCT/AU2014/000857

§ 371 Date: Mar. 3, 2016

(30) Foreign Application Priority Data
Sep. 3, 2013 (AU) 2013903349

(51) Int. Cl. C12N 15/82; C12N 9/10

(52) U.S. Cl. CPC C12N 15/8273; C12N 9/1051; C12N 15/8261; C12Y 204/01017

(57) ABSTRACT

Plants and plant cells derived therefrom are disclosed having one or more improved or introduced desirable traits such as increased plant biomass including both increased shoot and root growth and increased seed yield. Also disclosed is a method for the production of plants with increased cold tolerance, salt tolerance, drought tolerance, and resistance to parasitic plants. Plants can be produced which have an exogenous polynucleotide molecule encoding a glycosyltransferase (UGT) and/or an exogenous polynucleotide molecule which increases transcription of an endogenous UGT.

### TECHNICAL FIELD

The present invention relates to the production of plants having one or more improved or introduced desirable traits such as growth rate and/or yield, cold tolerance, salt tolerance, drought tolerance, and resistance to parasitic plants. In a particular application of the invention, plants are produced which comprise an exogenous polynucleotide molecule encoding a uridine diphosphate glucose (UDP) glycosyltransferase (UGT) and/or an exogenous polynucleotide molecule which increases transcription of an endogenous gene encoding a UGT.

### BACKGROUND

The desiccation tolerant grass Sporobolus stapfianus grows in arid, nutrient poor regions where it experiences short, sporadic rainfall events. In order to survive, the plant relies on an ability to desiccate (loss of ≧95% total water content) and rehydrate rapidly. The plant is also able to restore normal metabolism within 24 hours and grows very quickly following rain. Due to these characteristics, the plant has been much studied and, indeed, it has proven to be an excellent tool for pinpointing genes for increased stress-tolerance (Neale et al., 2000) as well as enhanced growth rate and nutrient acquisition (Blomstedt et al., 2010). As a result of this work, several novel Sporobolus drought genes (SDGs) have been identified that are specifically expressed in desiccation-tolerant tissue in dried S. stapfianus plants (Le et al., 2007). These SDGs have not, to date, been characterised in other plant species and their molecular functions remain unknown. However, their characterisation has the potential to reveal new molecular mechanisms for coping with severe stress that are peculiar to, or enhanced in, resurrection plants. Such coping mechanisms may include the ability to down-regulate and up-regulate growth rapidly in response to substantive changes in water status, to inhibit dehydration-induced senescence/cell death programs, to protect vital cellular components during dehydration and to reinstitute full photosynthetic capacity quickly following a severe dehydration event (Gaff et al., 2009; Blomstedt et al., 2010). Such mechanisms are likely to require coordinately regulated plant hormonal activity tightly linked to environmental cues.

The recently categorised class of plant hormones, strigolactones, have been shown to respond to environmental cues such as low nutrient conditions (eg nitrogen deficient and/or phosphorus deficient conditions) (Yoneyama et al., 2007), to interact with abscisic acid (ABA), and to be involved in growth related auxin and ethylene activity (López-Ráez et al., 2010; Koltai, 2011; Leyser, 2009; Dun et al., 2009). In some plant species, mutations in strigolactone (SL) production or perception pathway genes have been shown to delay flowering time, reduce senescence and decrease root mass (Woo et al., 2001; Snowden et al., 2005) and, as coordinators of shoot and root growth, strigolactones may have an important role, via cross-talk with other phytohormones, in the response of resurrection plants to alterations in environmental conditions.

Strigolactones are carotenoid-derived terpenoid lactones that are synthesised mainly, but not exclusively, in the roots of plants and can be transported in the xylem sap to the shoots (Kohlen et al., 2011). In the roots of many plants, strigolactones can act as stimulants of parasitic Striga and Orobanche plant germination and hyphal branching of symbiotic arbuscular mycorrhizal fungi. In Arabidopsis, strigolactones have been shown to regulate lateral root formation, root hair length and primary root growth in response to phosphate and/or carbohydrate availability (Kapulnik et al., 2011; Ruyter-Spira et al., 2011). It has been suggested that the effect of strigolactone on root architecture is mediated via a largely positive effect on ethylene and, subsequently, auxin biosynthesis, signaling and transport (Stepanova et al., 2009, Koltai, 2011). In the shoots, increased strigolactone levels coming from the roots, have been found to inhibit tiller formation or lateral bud outgrowth (Umehara et al., 2010; Kohlen et al., 2011) and mutations in the strigolactone biosynthesis and signaling pathway are known to often be associated with a hyperbranched, dwarfed phenotype (Dun et al., 2009; Leyser, 2009). Further, recent work indicates that strigolactones positively regulate vascular cambium growth, either independently or downstream of auxin signaling, leading to thicker stems and roots (Augusti et al., 2011). Accordingly, strigolactones have a major role in controlling plant architecture in response to environmental conditions.

One of the novel Sporobolus drought genes, namely SDG8i, whose molecular function has been under investigation by the present applicant, has now been found to encode a UDP-glycosyltransferase (UGT). The transcript levels of this UGT increase substantially under severe water deficit (Le et al., 2007). Plant genomes typically encode a large number of UGTs that conjugate sugars to a wide range of small acceptor molecules including many plant hormones, secondary metabolites and xenobiotics. As such, UGTs have an important role in cellular metabolism since glycosylation can affect the solubility, transport and biological activity of plant hormones and other acceptors (Lim et al., 2004). In experimentation described hereinafter, the present applicant has found that expression of SDG8i in the model plant species Arabidopsis, has a profound effect on plant architecture and growth. In particular, it has been found that SDG8i encodes a UGT that glycosylates one or more strigolactone or strigolactone-like compound and expression of this SL-UGT can enhance plant growth and/or stress responses.

### SUMMARY

Thus, in a first aspect, the present invention provides a plant cell of, or derived from, a plant comprising an exogenous polynucleotide molecule encoding a uridine diphosphate glucose (UDP) glycosyltransferase (UGT) and/or an exogenous polynucleotide molecule which increases transcription of an endogenous gene encoding a UGT, wherein said UGT is a UGT which glycosylates one or more strigolactone or strigolactone-like compound (ie an SL-UGT).

The plant cell may form part of a multicellular structure such as a whole plant, plant tissue, a component or organ of a plant, and reproductive material thereof (eg viable seed). Preferably, the plant cell is of the species Brassica napus (canola).

In a second aspect, the present invention provides a method of producing a plant with one or more improved or introduced desirable traits, said trait(s) being selected from the group consisting of growth rate and/or yield (eg increased seed/grain production), cold tolerance, salt tolerance, drought tolerance, and resistance to parasitic plants, said method comprising introducing to said plant an exogenous polynucleotide molecule encoding a uridine diphosphate glucose (UDP) glycosyltransferase (UGT) and/or an exogenous polynucleotide molecule which increases transcription of an endogenous gene encoding a UGT.

In a further aspect, the present invention provides a product of a whole plant according to the invention such as, for example, a harvested product such as grain or straw, or a processed product such as syrup or fibre. Thus, where the whole plant is an oilseed (eg B. napus (canola)), products encompassed by the invention include canola seed and oil.

### BRIEF DESCRIPTION OF FIGURES

FIG. 1 provides graphical results showing catalytic function of the enzyme encoded by the SDG8i (ie SL-UGT), as present in leaf protein extracts of N. benthamiana infiltrated with a CaMV 35S promoter driven SDG8i construct, tested against a number of substrates (known ability to affect plant growth and development) and compared with activity in extracts infiltrated with a vector-only control (A). The SDG8i extract showed substantial glycosylation activity of the synthetic strigolactone analog GR24 with a Km of 0.349 mM and a Vmax of 5.67 (B). No activity above background endogenous NADH oxidase was observed with any of the other substrates tested;

FIG. 2 provides graphical results showing germination rates of Orobanche seeds by SDG8i (ie SL-UGT) transgenic Arabidopsis plants, WT Arabidopsis Columbia plants, sorghum and S. stapfianus seedlings in vitro. The percent germination was calculated by counting the number of seeds having an emerged radicle. Values shown are the means±SE of 5 replicates;

FIG. 3 provides images of wild-type (WT) Arabidopsis Columbia plants and SL-UGT transgenic Arabidopsis plants growing at 21° C. under long day (LD) (A) and short day (SD) (B) photoperiods, showing increased height and branching of the SDG8i transgenic plants;

FIG. 4 provides graphical results of the primary bolt height (A) and inflorescence formation (B) in seven SL-UGT transgenic Arabidopsis plants and WT Arabidopsis Columbia plants. The plants were grown at 21° C. under LD conditions after stratification. The height and the number of inflorescences were measured 35 days after germination. Values shown are the means±SE of 5 replicates;

FIG. 5 provides graphical results of the measured shoot biomass (FW) of SL-UGT transgenic Arabidopsis plants and WT Arabidopsis Columbia plants growing in LD conditions. The biomass was measured 16 days after germination and before bolting. Values shown are the means±SE of 4 replicates;

FIG. 6 provides graphical results showing the growth parameters of SL-UGT transgenic Arabidopsis plants and WT Arabidopsis Columbia plants: (A) number of leaves, (B) leaf length, (C) number of inflorescences, and (D) plant height. The plants were grown at 21° C. under SD conditions following stratification, and measurements taken 30 days after bolting. Values shown are the means±SE of 5 replicates;

FIG. 7 graphically shows the seed yield from each of seven SL-UGT transgenic Arabidopsis plants and WT Arabidopsis Columbia plants grown at 21° C. under SD conditions after stratification. Dry seeds were collected following senescence. Values shown are the means±SE of 5 replicates;

FIG. 8 provides graphical results of: (A) the measurement of primary root length in each of seven SL-UGT transgenic Arabidopsis plants and WT Arabidopsis Columbia plants grown in the dark at 21° C. for 7 days after stratification. Values shown are the means±SE of 5 replicates; and (B) the root biomass (FW) of pre-flowering SL-UGT transgenic Arabidopsis plants and WT Arabidopsis Columbia, measured 16 days after germination grown in LD at 21° C. Values shown are the means±SE of 4 replicates;

FIG. 9 shows the effect of SL-UGT expression on the length of primary root (A), and of fully elongated cortical cells (B), lateral root primordium density (C), and lateral root initiation index (D) in SL-UGT transgenic Arabidopsis plants and WT Arabidopsis Columbia plants. Plants were grown in vertical orientation at 21° C. under SD following stratification for 13 days in MS media. Values shown are the means±SE of 4 replicates;

FIG. 10 provides graphical results showing the effect of salt on the root growth of five SL-UGT transgenic Arabidopsis plants and WT Arabidopsis Columbia plants. The plants were grown upright for 4-6 days in MS media at 21° C. under SD conditions following stratification and then transferred to media containing different levels of NaCl and the plates inverted. Root growth (cm) was measured after 7 days. The values shown are the means±SE of 10 replicates;

FIG. 11 provides images of wild-type (WT) Arabidopsis Columbia plants and SL-UGT transgenic Arabidopsis plants (T) on day 7 and day 13 of a 14-day waterholding period, and at day 15, 24 hours following re-watering;

FIG. 12 shows (A) the protoplasmic drought tolerance (PDT) survival curve of three SL-UGT transgenic Arabidopsis plants and WT Arabidopsis Columbia plants, and indicates the effect of decreasing relative humidity on cell survival; and (B) the leaf survival curve of the same three SL-UGT transgenic Arabidopsis plants and WT Arabidopsis Columbia plants. The leaf survival curve shows the effect of decreasing relative humidity;

FIG. 13 provides images of wild-type (WT) Arabidopsis Columbia plants, SL-UGT transgenic Arabidopsis plants (SDG8i) and transgenic Arabidopsis plants expressing SDG10y and SDG7y growing at 21° C. under short day (8 h day/16 h night) after stratification;

FIG. 14 provides graphical results of different growth parameters for SDG10y- and SDG7y-expressing transgenic plants, and WT Arabidopsis Columbia plants. The plants were grown at 21° C. under the long day condition of 16 h day/8 h night. Inflorescence (A) and height (B) were measured 27 days and 38 days after germination, respectively. Values are the means 4 SE of 8 replicates; and

FIG. 15 provides graphical results of different growth parameters for SDG10y- and SDG7y-expressing transgenic plants, and WT Arabidopsis Columbia plants. The plants were grown at 21° C. under the short day condition of 16 h day/8 h night. Inflorescence (A) and height (B) were measured 48 days and 40 days after bolting, respectively. Values are the means±SE of 8 replicates.

### DETAILED DESCRIPTION

In work leading to the present invention, the applicant had previously isolated cDNA and genomic sequences for a desiccation-induced gene, designated SDG8i, from the perennial, resurrection grass S. stapfianus (Le et al., 2007; the disclosure of which is herein incorporated by reference). The nucleotide sequence comprising the coding region of SDG8i is shown as SEQ ID NO: 1, while the nucleotide sequence of the full-length structural gene is shown as SEQ ID NO: 2. The structural gene contains no introns and encodes a protein of 473 amino acids with a calculated molecular mass of 52 kDa. The amino acid sequence of the protein encoded by SDG8i is shown as SEQ ID NO: 3. Database analysis has revealed that the encoded protein is a glycosyltransferase (GT) belonging to Family 1; which is characterised by a putative plant secondary product glycosyltransferase (PSPG) motif in the C-terminal domain. Moreover, by comparison with amino acid sequences of related proteins from other plants, including maize, Arabidopsis, tomato and tobacco, and the identification of the presence of key amino acid residues for donor sugar recognition (cf Glu-381 and GLn-382 in the PSPG motif of the Medicago truncatula glucosyltransferase), the protein encoded by SDG8i has been identified as a uridine diphosphate glucose (UDP) glycosyltransferase (UGT). As it was subsequently recognised that UGTs have an important role in the cellular metabolism of plants, since glycosylation can affect the solubility, transport and biological activity of plant hormones and other acceptors, the present applicant sought to assess whether the SDG8i gene could be used to confer desirable traits such as plant growth and/or stress responses in other plant species. Using the model plant species Arabidopsis, it was found that expression of SDG8i in this species could enhance plant growth, seed yield, resistance to parasitic plants, and increased tolerance to drought, salt and cold, all without having any adverse effects on plant development or morphology.

Thus, in a first aspect, the present invention provides a plant cell of, or derived from, a plant comprising an exogenous polynucleotide molecule encoding a uridine diphosphate glucose (UDP) glycosyltransferase (UGT) and/or an exogenous polynucleotide molecule which increases transcription of an endogenous gene encoding a UGT, wherein said UGT is a UGT which glycosylates one or more strigolactone or strigolactone-like compound (ie an SL-UGT).

The plant cell of the present invention expresses an exogenous UGT and/or an increased amount of an endogenous UGT. As will be appreciated by persons skilled in the art, the expression of exogenous UGT, as well as the expression of an increased amount of an endogenous UGT, may be regarded as “overexpression” of UGT.

The term “strigolactone” as used herein, will be understood by persons skilled in the art as referring to the class of carotenoid-derived terpenoid lactones, generally having a chemical structure according to;

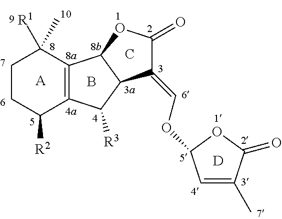

Such compounds are known to modulate the activity of F-box proteins such as the MAX2 protein found in Arabidposis and related proteins encoded by orthologous genes in other species. As used herein, “strigolactone-like compounds” is to be understood as referring to structurally related compounds that may similarly modulate the activity of F-box proteins and/or represent intermediate compounds in the strigolactone synthesis pathway.

It is generally accepted that strigolactone and/or strigolactone-like compounds are present in more than 90% of land plants. Consequently, the plant cell of the present invention may be selected from a wide variety of species including, for example, crop and forage species of plants such as:
Maize: eg Zea mays,
Wheat: eg Triticum aestivum, T. spelta, T. durum,
Medics: eg Medicago truncatula, M. littoralis,
Rice: eg Oryza sativa, O. glaberrima,
Sorghum: eg Sorghum bicolor,
Sugarcane: eg Saccharum officinarum,
Oilseed: eg Brassica napus (canola), Brassica campestris,
Soybean: eg Phaseolus max, and
Cotton: eg Gossypium hirsutum,
   and hybrids thereof, as well as species and hybrids of lawn grasses (eg Lolium spp., Festuca spp., and Cynodon spp.) and ornamental plants, particularly those requiring substantially continual moist conditions (eg camelias, rhododendrons and ferns).

Preferably, the plant cell forms part of a multicellular structure such as a whole plant (including, for example, seedlings and mature plants), but including plant tissue (including a plant tissue culture), a component or organ of a plant, and reproductive material thereof (eg viable seed).

Preferably, the exogenous polynucleotide molecule(s) is/are stably integrated into the plant cell's genome. Such plant cells may be obtained, for example, from a plant transformed by using any of the methods of plant tranagenesis well known to persons skilled in the art such as those described by Slater et al. (2003) and Christou et al. (2004), and methods involving, for example, Agrobacterium infection, microinjection of the polynucleotide molecule(s) into plant cells, electroporation and the use of so-called gene guns or microbombardment, as well as receptor mediated transformation and the use of viral vectors. The plant cell may be hemizygous or homozygous for the exogenous polynucleotide molecule(s); that is, the plant cell may have an exogenous polynucleotide molecule present at a single locus in the genome (hemizygous) or, more preferably, has a copy of the exogenous polynucleotide molecule at the same locus on each chromosome of a chromosome pair (homozygous). Homozygous transgenic plants can be obtained by, for example, “selfing” a hemizygous plant.

Particular methods of plant transgenesis have been described for maize (eg Scott 2009), wheat (eg reviewed by Abdul et al., 2010), annual medics (eg Chabaud et al., 1995), rice (eg Ho et al., 2001), sorghum (eg Casas et al., 1993), sugarcane (eg Arencibia et al., 1992), oilseed (eg El-Awady et al., 2008), soybean (eg Rech et al., 2008), cotton (eg Cousins et al., 1991), lawn grasses (eg reviewed by Wang et al., 2006) and ornamental plants (eg reviewed in JAT da Silva (ed)). The disclosure of each of the publications referred to in this paragraph is incorporated herein by reference.

In embodiments where the plant cell comprises an exogenous polynucleotide molecule encoding UGT, the UGT is preferably a SL-UGT comprising an amino acid sequence that has at least 35%, preferably at least 50%, more preferably at least 70%, still more preferably at least 90%, and yet more preferably, at least 98% sequence identity to the amino acid sequence shown as SEQ ID NO: 3 or a biologically active fragment thereof. Most preferably, the UGT is a SL-UGT comprising an amino acid sequence that is identical or substantially identical to the sequence shown as SEQ ID NO: 3 or a biologically active fragment thereof. Biologically active fragments of the amino acid sequence shown as SEQ ID NO: 3 may be determined using any of the suitable methods well known to persons skilled in the art, and may involve recombinant expression or synthesis of variously truncated forms of the full-length protein and assaying for SL-UGT activity (for example, as described in Example 1 herein). Such biologically active fragments may comprise the PSPG region and the amino acid residues responsible for binding a strigolactone or strigolactone-like compound.

Percentage values of amino acid sequence identity given herein are calculated using the NCBI algorithm, basic local alignment search tool for protein sequences (blastp).

The term “substantially identical” as used herein in relation to an amino acid sequence, is to be understood as encompassing minor variations in that particular amino acid sequence which do not result in any significant alteration of the biological activity of its derivative protein, polypeptide or peptide. These variations may include conservative amino acid substitutions. Exemplary conservative amino acid substitutions are provided in Table 1 below.

TABLE 1
Exemplary conservative amino acid substitutions

| | Conservative Substitutions |
|---|---|
| Ala | Val*, Leu, Ile |
| Arg | Lys*, Gln, Asn |
| Asn | Gln*, His, Lys, Arg, Asp |
| Asp | Glu*, Asn |
| Cys | Ser |
| Gln | Asn*, His, Lys, |
| Glu | Asp*, γ-carboxyglutamic acid (Gla) |
| Gly | Pro |
| His | Asn, Gln, Lys, Arg* |
| Ile | Leu*, Val, Met, Ala, Phe, norleucine (Nle) |
| Leu | Nle, Ile*, Val, Met, Ala, Phe |
| Lys | Arg*, Gln, Asn, ornithine (Orn) |
| Met | Leu*, Ile, Phe, Nle |
| Phe | Leu*, Val, Ile, Ala |
| Pro | Gly*, hydroxyproline (Hyp), Ser, Thr |
| Ser | Thr |
| Thr | Ser |
| Trp | Tyr |
| Tyr | Trp, Phe*, Thr, Ser |
| Val | Ile, Leu*, Met, Phe, Ala, Nle |

*indicates preferred conservative substitutions

Particular conservative amino acid substitutions envisaged are:
Substitution between the following amino acids: Gly, Ala, Val, lie, Leu and Met;
Substitution between the following amino acids: Asp and Glu;
Substitution between the following amino acids: Asn and Gin;
Substitution between the following amino acids: Ser and Thr;
Substitution between the following amino acids: Lys, Arg and His;
Substitution between the following amino acids: Phe, Tyr, Trp and His; and
Substitution between the following amino acids: Pro and Nα-alkylamino acids.

The exogenous polynucleotide molecule encoding UGT may be derived from a plant. Preferably, such an exogenous polynucleotide molecule is derived from a resurrection plant (eg Anastatica hierochuntica (rose of Jericho), plants of the genus Asteriscus or Mesembryanthemum, and Selaginella lepidophylla (stone flower)) or, more preferably, a resurrection grass (eg S. stapfianus, Oropetium thomaeum and Micraira sp).

As mentioned above, the UGT is one which glycosylates one or more strigolactone or strigolactone-like compound and, most preferably, is a SL-UGT comprising an amino acid sequence that is identical or substantially identical to the sequence shown as SEQ ID NO: 3 or a biologically active fragment thereof. It is considered that orthologues of nucleotide sequences encoding an SL-UGT comprising the amino acid sequence shown as SEQ ID NO: 3 exist in a variety of plant species, including Hordeum vulgare (barley) and Arabidopsis species. As will be appreciated by persons skilled in the art, such orthologues are suitable for use in the present invention.

The exogenous polynucleotide molecule encoding UGT may be introduced in a manner whereby expression of the UGT is driven by expression control elements (eg promoters and enhancer elements etc) that are endogenous to the plant cell (eg the exogenous polynucleotide molecule may be integrated into the genome of the plant cell at a site where expression is driven by an existing, endogenous expression control element), or which are exogenous to the plant cell (eg the exogenous polynucleotide sequence may comprise, in addition to the UGT-coding sequence, an operably linked expression control element(s), wherein said expression control element(s) may be native to the plant cell (ie the expression control element(s) may comprise at least one element, such as a promoter sequence, that is derived from the species of the plant cell) but more preferably, will be foreign to the plant cell but nevertheless functional in the plant cell. The foreign control element(s) may be derived from a gene from which a UGT-coding sequence has been derived, or may be foreign to the UGT-coding sequence. For example, the UGT-coding sequence may be operably linked to a foreign promoter sequence and/or a cis-acting enhancer or other transcription regulatory sequence, by any of the suitable methods well known to persons skilled in the art. Conveniently, the UGT-coding sequence may be introduced into an expression cassette or expression vector such that the coding sequence is operably-linked to a promoter sequence provided by said cassette or vector. Suitable promoter sequences for expression of the UGT-coding sequence may be inducible or constitutive promoters and may drive expression, within for example a whole plant, ectopically, throughout the whole plant or in a tissue-specific or differential manner. Preferably, a promoter sequence will be selected that is a constitutive promoter (eg a strong constitutive promoter such as the well-known CaMV35S promoter and the 2×35S promoter).

In embodiments where the exogenous polynucleotide encoding UGT is expressed in a tissue-specific or differential manner, the expression may occur in any tissue of the plant for which a suitable promoter has been identified, as persons skilled in the art would appreciate. The nature of the tissue-specific promoters to be utilised may vary with the plant species, for example, gene expression may be differential expression rather than truly tissue-specific for some plant species. Preferably, the tissue-specific or differential expression occurs in the roots and/or shoots of the plant. In an embodiment, the cauliflower mosaic virus 35S (CaMV35S) promoter (eg the −343 to +8 bp region) can be utilised to drive high-level expression in a number of plant species. However, the CaMV35S promoter consists of two domains, Domain A and Domain B (Benfey et al., 1989). Domain A (the −90 to +8 bp region) contains the activation sequence 1 (as-1) regulatory element and may be utilised to drive gene expression in root tissue. Domain B (the −343 to −90 bp region) may be utilised to drive expression in shoot tissue. Alternatively, tissue-specific promoters may be derived directly from genes endogenous to the plant species by isolating genes expressed in a specific tissue using methods known in the art, such as differential screening. For example, the Arabidopsis acidic chitinase promoter and the root promoter, EIR1, have been shown to have differential root/shoot expression (Samac & Shah, 1991; Luschnig et al., 1998). Promoters can also be synthesised using information on tissue-specific regulatory elements contained in gene expression databases. For example, the GENEvestigator Arabidopsis microarray database provides information on the tissue/organ specificity of gene expression (Hruz et al., 2008). Persons skilled in the art will appreciate that other promoters or promoter domains can be utilised to drive gene expression in particular tissues.

In embodiments where the plant cell comprises an exogenous polynucleotide molecule which increases transcription of an endogenous gene encoding a UGT, the exogenous polynucleotide molecules may encode one or more transcription factors that increase transcription (and, consequently, expression) of an endogenous gene encoding a UGT. Such polynucleotide molecules may be derived from endogenous transcription factor regulatory genes (ie such that the transcription factor(s) will be native to the plant cell, but expressed in, for example, a constitutive manner and/or at an increased level). However, preferably, such polynucleotide molecules are derived from transcription factor regulatory genes from an exogenous source (ie such that the transcription factor(s) will be foreign to the plant cell but nevertheless functional in the plant cell). Suitable transcription factor regulatory genes may be identified using standard methods well known to persons skilled in the art such as the yeast one-hybrid system (Ouwerkerk and Meijer, 2001).

An exogenous polynucleotide molecule which increases transcription of an endogenous gene encoding an SL-UGT, may preferably encode an SDG10y and/or SDG7y ERF transcription factor(s) (Mizoi et al., 2012). The nucleotide sequences of polynucleotide molecules encoding the SDG10y and/or SDG7y transcription factors of S. stapfianus are shown as SEQ ID NO: 4 and SEQ ID NO: 5 respectively, while the amino acid sequences of those transcription factors are shown as SEQ ID NO: 6 and SEQ ID NO: 7.

In embodiments where the plant cell comprises an exogenous polynucleotide molecule which encodes one or more transcription factor, the transcription factor is preferably one comprising an amino acid sequence that has at least 60%, more preferably at least 70%, still more preferably at least 90%, and yet more preferably, at least 98% sequence identity to the amino acid sequence shown as SEQ ID NO: 6 (or a biologically active fragment thereof) or SEQ ID NO: 7 (or a biologically active fragment thereof). Most preferably, the transcription factor is one comprising an amino acid sequence that is identical or substantially identical to the sequence shown as SEQ ID NO: 6 (or a biologically active fragment thereof) or SEQ ID NO: 7 (or a biologically active fragment thereof). Suitable exogenous polynucleotide molecules encoding a transcription factor which increases transcription of an endogenous gene encoding an SL-UGT, may be derived from a plant such as a resurrection plant or, more preferably, a resurrection grass. Orthologues of nucleotide sequences encoding the transcription factors comprising the amino acid sequence shown as SEQ ID NO: 6 or SEQ ID NO: 7 are likely to be found in a variety of plant species, including Arabidopsis species and grass species such as Z. mays and O. sativa. As will be appreciated by persons skilled in the art, such orthologues are suitable for use in the present invention.

An exogenous polynucleotide molecule encoding a transcription factor which increases transcription of an endogenous gene encoding an SL-UGT, may be introduced into the plant cell in a manner whereby expression of the transcription factor is driven by expression control elements that are endogenous to the plant cell (eg the exogenous polynucleotide molecule may be integrated into the genome of the plant cell at a site where expression is driven by an existing, endogenous expression control element), or which are exogenous to the plant cell. For example, the exogenous polynucleotide sequence may comprise, in addition to the transcription factor-coding sequence, an operably linked expression control element(s), wherein said expression control element(s) may be native to the plant cell, but more preferably, will be foreign to the plant cell but nevertheless functional in the plant cell. The foreign control element(s) may be derived from a gene from which the transcription factor-coding sequence has been derived, or may be foreign to the transcription factor-coding sequence. For example, the transcription factor-coding sequence may be operably linked to a foreign promoter sequence and/or a cis-acting enhancer or other transcription regulatory sequence. Conveniently, the transcription factor-coding sequence may be introduced into an expression cassette or expression vector such that the coding sequence is operably-linked to a promoter sequence provided by said cassette or vector. Suitable promoter sequences for expression of the transcription factor-coding sequence may be inducible or constitutive promoters and may drive expression, within for example a whole plant, ectopically, throughout the whole plant or in a tissue-specific manner. Preferably, a promoter sequence will be selected that is a constitutive promoter such as CaMV35S promoter or 2×35S promoter mentioned above.

In some multicellular embodiments of the invention, the exogenous UGT and/or endogenous UGT is preferably expressed in all cells (eg throughout a whole plant, plant tissue, plant component or organ, or reproductive material thereof). Preferably, such expression is also constitutive. As indicated above, expression of an exogenous UGT and/or endogenous UGT resulting in, for example, constitutive glycosylation of one or more strigolactone or strigolactone-like compound, can enhance plant growth (eg growth rate to thereby provide higher yields) and/or stress responses (eg to improve tolerance to cold, salt and drought stress). Further, in the model plant species Arabidopsis, it appears that the expression of an exogenous UGT and/or endogenous UGT does not substantially affect homeostasis since plant architecture and developmental rate is not adversely affected, thereby indicating that phytohormones are maintained in balance. Moreover, while not wishing to be limited by theory, it appears that the effect of the expression of an exogenous UGT and/or endogenous UGT is an increase in the procurement/availability of resources (possibly through enhanced root growth) or increased nutrient-use efficiency, or both, leading to a more robust productive plant. In addition, it has been observed in the model plant species Arabidopsis, that the expression of an exogenous UGT and/or endogenous UGT can also lead to reduced stimulation of parasitic seed germination that is, these plants show increased resistance to parasitic plants such of the genus Orobanche (broomrape; eg O. ramosa and O. uniflora) and Striga (“witches weed”; eg. S. asiatica, S gesnerioides and S. hermonthica). Such parasitic plants can cause significant crop losses in, for example, maize, sorghum and sugarcane.

In a second aspect, the present invention provides a method of producing a plant with one or more improved or introduced desirable traits, said trait(s) being selected from the group consisting of growth rate and/or yield, cold tolerance, salt tolerance, drought tolerance, and resistance to parasitic plants, said method comprising introducing to said plant an exogenous polynucleotide molecule encoding a uridine diphosphate glucose (UDP) glycosyltransferase (UGT) and/or an exogenous polynucleotide molecule which increases transcription of an endogenous gene encoding a UGT, wherein said UGT is a UGT which glycosylates one or more strigolactone or strigolactone-like compound (ie an SL-UGT).

A plant produced in accordance with the method of the second aspect will show overexpression of UGT, wherein the expression is, preferably, throughout the plant. The plant may be selected from, for example, crop and forage species and hybrids thereof, species and hybrids of lawn grasses, and ornamental plants as described above. Preferably, the exogenous polynucleotide molecule(s) is/are stably integrated into the plant's genome. Further, the plant is preferably homozygous for the exogenous polynucleotide molecule(s).

In some embodiments, a plant produced in accordance with the method will show improved growth rate and/or yield (eg increased seed/grain production) relative to an identical plant lacking said exogenous polynucleotide molecule(s) grown under equivalent conditions.

In some embodiments, a plant produced in accordance with the method will show improved cold tolerance relative to an identical plant lacking said exogenous polynucleotide molecule(s) grown under equivalent conditions. Such growth conditions may comprise challenge by cold conditions (eg such as that used in standard freezing stress assays, an example of which is described in Example 1 hereinafter).

In some embodiments, a plant produced in accordance with the method will show improved salt tolerance relative to an identical plant lacking said exogenous polynucleotide molecule(s) grown under equivalent conditions. Such growth conditions may comprise challenge by salt stress conditions (eg such as that used in standard salt stress assays, an example of which is described in Example 1 hereinafter).

In some embodiments, a plant produced in accordance with the method will show improved drought tolerance relative to an identical plant lacking said exogenous polynucleotide molecule(s) grown under equivalent conditions. Such growth conditions may comprise challenge by drought stress conditions (eg such as that used in standard drought stress assays, including, for example, simple water witholding assays and water vapour equilibration assays such as those described in Example 1 hereinafter).

In some embodiments, a plant produced in accordance with the method will show improved resistance to parasitic plants (eg parasitic plants of the genus Orobanche and Striga) relative to an identical plant lacking said exogenous polynucleotide molecule(s) grown under equivalent conditions. Such growth conditions may comprise challenge by one or more parasitic plants (eg as performed in a standard Orobanche germination assay).

In a further aspect, the present invention provides a product of a whole plant according to the invention such as, for example, a harvested product such as grain or straw, or a processed product such as syrup or fibre. Thus, where the whole plant is an oilseed (eg B. napus (canola)), products encompassed by the invention include canola seed and oil.

In order that the nature of the present invention may be more clearly understood, preferred forms thereof will now be described with reference to the following non-limiting example.

### EXAMPLES

### Example 1

### SDG8i Introduced into Arabidopsis Enhances Growth Rate and Stress Tolerance

### Materials and Methods

### Orobanche Germination Assay

Orobanche seeds were surface sterilised with 2% sodium hypochlorite containing 0.02% (v/v) of polysorbate 20 surfactant (Tween 20™; Sigma-Aldrich Co LLC, St Louis, Mo., United States of America) for 5 min then rinsed (5×) with sterile ddH₂O and dried for 30 min in a laminar air flow cabinet. Subsequently, between 50 and 100 seeds were spread on a sterile 9-mm glass fibre disk and placed in petri dishes (9 cm diameter) lined with filter paper wetted with 3 mL of ddH₂O. 1 ml of gibberellic acid (GA3) (30 mg/L) was applied to the seeds and the petri dishes then sealed with parafilm and incubated at 18-20° C. in the dark for preconditioning. After 7 days, surface-sterilised seeds of Arabidopsis W/T, SDG8i transgenics, sorghum and & stapfianus were placed on 5 cm glass microfiber disks and incubated on ¼ MS media with 0.5% sugar and 0.8% agar. For controls, 0.6 ml aqueous solution of synthetic strigolactone analog GR24 (Chiralix B.V., Toernooiveld 1006525 EC Nijmegen, The Netherlands) (0.0001 mg/1) or sterile water was applied to petri dishes with preconditioned Orobanche seeds. The plates were then resealed with parafilm and wrapped with foil and incubated at 18-20° C. for 7 days for germination. Orobanche seed germination was determined under a stereoscopic microscope by counting the number of seeds having an emerged radicle.

### Recombinant UGT Production

UGT was produced in N. benthamiana leaf tissue by transient transfection using a viral-based MagnICON vector system (Icon Genetics GmbH, Halle/Salle, Germany).

Briefly, the UGT coding sequence was amplified from the SDG8i cDNA (Le et al., 2007) and ligated into the MagnICON plasmid. The plasmid was then cultured in Escherichia coli DH5α (ATCC Catalog No 53868) and electroporated into Agrobacterium tumefaciens LBA4404). Subsequently, Nicotiana benthamiana leaves were covered in aluminium foil for 3 days before being co-infiltrated with the plasmid and the apoplast-targeting and intergrase modules of the MagnICON system (Marillonnet et al. 2005). Leaves were then harvested 5 days after the co-infiltration and homogenised in ice-cold protein extraction buffer. Following removal of the insoluble material by centrifugation at 13,000 rpm at 4° C. for 5 minutes, protein assays were carried out with Bio-Rad Protein Assay Dye (Bio-Rad Laboratories Inc, Hercules, Calif., United States of America) using bovine serum albumin (BSA) as reference. Protein extracts were analysed by SDS-PAGE using the standard methods described by Sambrook et al. (1989).

### UGT Enzyme Assay

UGT activity was measured using a coupled assay with pyruvate kinase and lactate dehydrogenase with the reaction rate monitored by the change in NADH absorbance at 340 nm (Jackson et al. 2001). Activity in millikatals kg⁻¹ was calculated using the extinction coefficient 6.22×10⁻³ M⁻¹ cm⁻¹ for NADH. The background activity of the extracts, monitored by the measurement of the rate without substrate addition, was subtracted from the reaction rate.

### Generation of Transgenic Plants

Gene specific primers were used to amplify the coding region of SDG8i using the Pfu proofreading polymerase. To allow the use of the Gateway cloning system (Invitrogen Corporation, Carlsbad, Calif., United States of America), the sequences for the recombination sites, attB1 and attB2, of the donor vector pDONR221 were included as part of the primers. Following successful generation of the pDONR221:SDG8i construct, the SDG8i coding sequence was then transferred to the plant expression vector pMDC32 containing the 2×35S promoter (Curtis et al., 2003). This construct was then introduced into Arabidopsis using A. tumifaciens (AgIi strain) by the floral dip method (Martinez-Trujillo et al., 2004). Transgenic plants were selected for resistance to hygromycin and taken through to the second generation (T2) to generate plants homozygous for SDG8i.

### Observation of Palisade Cells in Fully Expanded Leaves

Leaves were immersed in 0.1% Triton X-100, followed by centrifugation at 10,000 g for 1 min at room temperature (RT) to remove air bubbles from intercellular spaces and sediment the chloroplasts. Leaves were viewed under a light microscope. The area, length and width of the leaf blade was measured; the number and area of palisade cells were determined in the sub-epidermal layer, and the number of palisade cells aligned along the proximo-distal (P-D) and medio-lateral (M-L) axes were counted. Then the leaf index (leaf blade length/width ratio) and total cell number were calculated More particularly, leaves were taken from 25-day-old soil-grown plants grown under short day (SD) photoperiod conditions. For each leaf, the average cell area was determined by measuring 20 palisade cells within a 0.4 μm² area. The total number of palisade cells was determined by dividing the leaf area by the palisade cell density for each leaf.

### Examination of Root Architecture

Plants were grown at 21° C. under LD (8 h day/16 h night) following stratification for 13 days in vertically orientated petri dishes on MS media (pH5.8) with 1% sucrose and 0.8% agar. Roots were then fixed in 4% formaldehyde prepared in 0.025 M phosphate buffer (pH 7.2) for at least 4 h at room temperature, or O/N at 4° C. The fixative was replaced with 30% (aq v/v) glycerol containing 2% (v/v) DMSO and left for 30 min at room temperature. Roots were then mounted in a clearing solution composed of 4.2 M NaI, 8 mM Na₂S₂O₃ prepared in 65% (v/v) glycerol supplemented with 2% (v/v) DMSO, and observed 1 hour after the sample preparation. Root primordia and root cell-length analyses were performed under the microscope.

### Salt Stress Assays

Seeds were surface-sterilised and stratified at 4° C. for 2-4 days to obtain uniform germination and sown on 0.5% sucrose MS media without salt. Following 4-6 days growth with the plates in a vertical orientation, the seedlings were transferred to salt (0, 50, 100, 125, 150 and 175 mM NaCl) containing germination media (MS, 0.5% sucrose, pH 5.7, 0.8% agar). The position of the root tips was marked immediately after transfer and the agar plates placed in vertical orientation and inverted to allow roots to grow downward in the shape of a hook. Root elongation was quantified by marking the position of the root tips at intervals over 7-10 days.

### Freezing Stress Assay

Plants (25 seedlings/plate) were grown on MS-agar (½ MS, 0.5% sucrose, pH 5.7, 0.8% agar) media for 3 weeks at 22° C. after stratification. Seedlings were cold-acclimated at −1° C. for 16 hours. The temperature was lowered by 2° C. per hour until it reached −4° C. The seedlings were then exposed to −8° C. for 1 hour and to −12° C. for a further hour. Plants were removed periodically at −4° C., −8° C. and −12° C. and transferred to 4° C. overnight for recovery. Plants were returned to 22° C. and survival was scored 7 days later. Plants that were bleached of chlorophyll and appeared white were scored as dead, while green plants were scored as having survived the freezing test.

### Water Withholding Test

WT and SDG8i transgenic lines were grown in separate 5 cm diameter pots placed on the same tray under optimal growth conditions in a controlled growth chamber under a 16 h light regime at 22° C. for 2 weeks. Plants were kept fully watered by sub-irrigation until the 6-7 leaf pre-flowering stage. Water was then withheld for a period of 14 days. The plants were observed over this period and after 14 days, the drought treated plants were re-watered and recovery was checked 24 hours later.

### Water Vapour Equilibration

The protoplasmic drought tolerance (PDT) of Arabidopsis plants was assessed by a water vapour equilibration technique using CaCl₂ solutions (Slatyer, 1955; Gaff and Carr, 1961). CaCl₂ permits a wide range of water potentials (ψ_{w}), down to 30% relative humidity (RH) in equilibrium with a saturated solution, to be imposed on the plants via the air phase. Pre-flowering plants were washed gently and soaked in water for 2 hours until turgid and blotted dry. For PDT determination, shoots were detached and the initial turgid weight recorded. Additional plant samples were also used to determine an initial turgid weight to dry weight ratio (TWtₒ/DWtₒ). Transgenic and wild-type shoots (four per line) were placed on mesh inside closed chambers above the CaCl₂ solutions, avoiding contact between the leaf and solution, and equilibrated to differing RH. Chambers containing CaCl₂ solutions at 98% (−2.8 MPa), 96% (−5.5 MPa), 94% (−8.4 MPa), 92% (−11.3 MPa), 90% (−14.4 MPa), 88% (−17.4 MPa) and 86% (−20.5 MPa) RH at 25° C. were used. The relative humidity of air in equilibrium with the CaCl2 solutions are calculated from freezing point depression data using the formula in Owen (1952). The chambers were kept in a closed insulator box in a constant temperature room at 20° C. for at least 3 days until equilibration was reached (ψ_{leaf}=ψₛₒₗᵤₜᵢₒₙ). After equilibration, the shoots were soaked in water for 24 hours to rehydrate and the final turgid weight (TWt_{F}) was recorded. The ratio TWt_{F}/TWtₒ was plotted against % RH, and the PDT was determined as the % RH at which 50% of the tissue is alive. The dry weight of each sample was taken to calculate TWt_{F}/DWt_{F} and the ratio of TWt_{F}/DWt_{F}/TWtₒ/DWtₒ was also calculated. This value was used to estimate the index of damage.

### Leaf Survival Test

For the leaf survival test, a subjective estimate of the number of leaves alive per plant was recorded by comparing crisp, springy healthy green leaves with flaccid, discoloured leaves. Neutral red uptake was used to check live versus dead leaf tissue and also to compare cell damage in live leaves of transgenic and WT plants. The methodology for the neutral red uptake test was as follows: 29 ml of CaCl₂ solution was added to 1 ml of neutral red stock solution and NaOH was added until the colour changed from purple red to orange red. Hand sections of leaves of about 2-3 cells thick were stained with the neutral red solution for 60 minutes under 100% RH to prevent the NR solution from drying and viewed under a microscope. Live cells exhibited an intense red colour in the vacuole while dead cells showed a diffuse light orange colour.

### Results

### SDG8i Glycosylates GR24 In Vitro

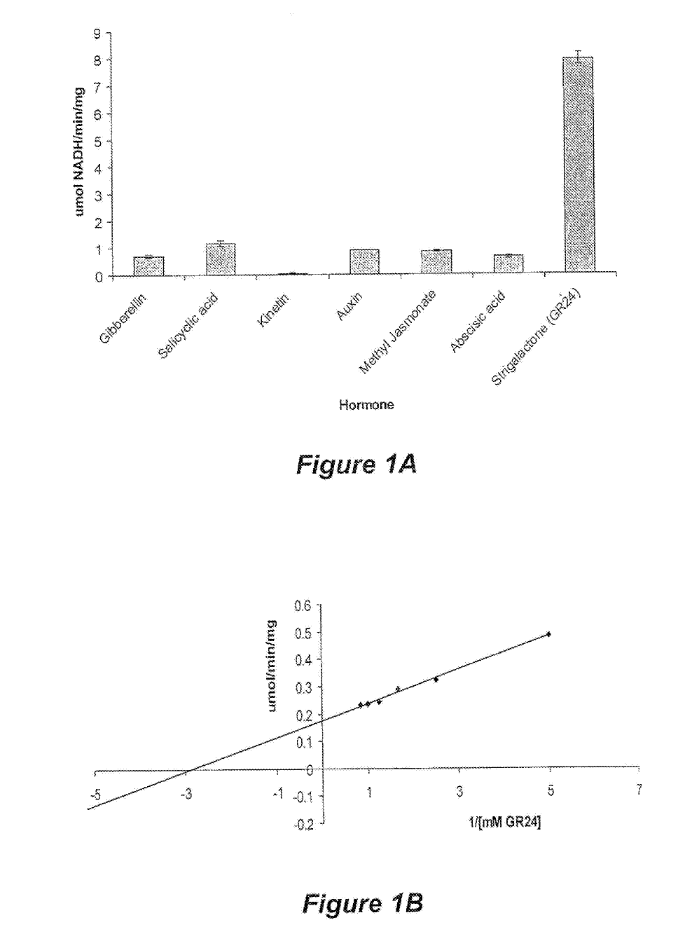

To investigate the catalytic function of the enzyme encoded by the SDG8i cDNA, the glucosyltransferase activity of N. benthamiana leaf protein extracts infiltrated with a CaMV 35S promoter driven SDG8i construct was tested against a number of substrates and compared with activity in extracts infiltrated with a vector-only control. The assay utilised a linked enzymic reaction as described in the Materials and Methods. The substrates were chosen for their known ability to affect plant growth and development. The results are shown in FIG. 1A; the SDG8i extract showed substantial glycosylation activity of GR24 with a Km of 0.349 mM and a Vmax of 5.67 (FIG. 1B). No activity above background endogenous NADH oxidase was observed with any of the other substrates tested. Also, the control extract showed no activity above background for any of the substrates. The results therefore indicated that SDG8i is capable of glycosylating the synthetic strigolactone analogue GR24.

### SDG8i Reduces the Stimulation of Orobanche Germination by Arabidopsis

The ability to glycosylate endogenous one or more strigolactone or strigolactone-like compounds in plant roots could affect the secretion of the stimulus signals required by parasitic plants to germinate. While Arabidopsis does not host arbuscular mycorrhizal fungi, the plant produces at least two strigolactones (orobanchol and orobanchyl acetate) and root exudates can stimulate Pinguicula ramosa germination (Kohlen et al. 2011).

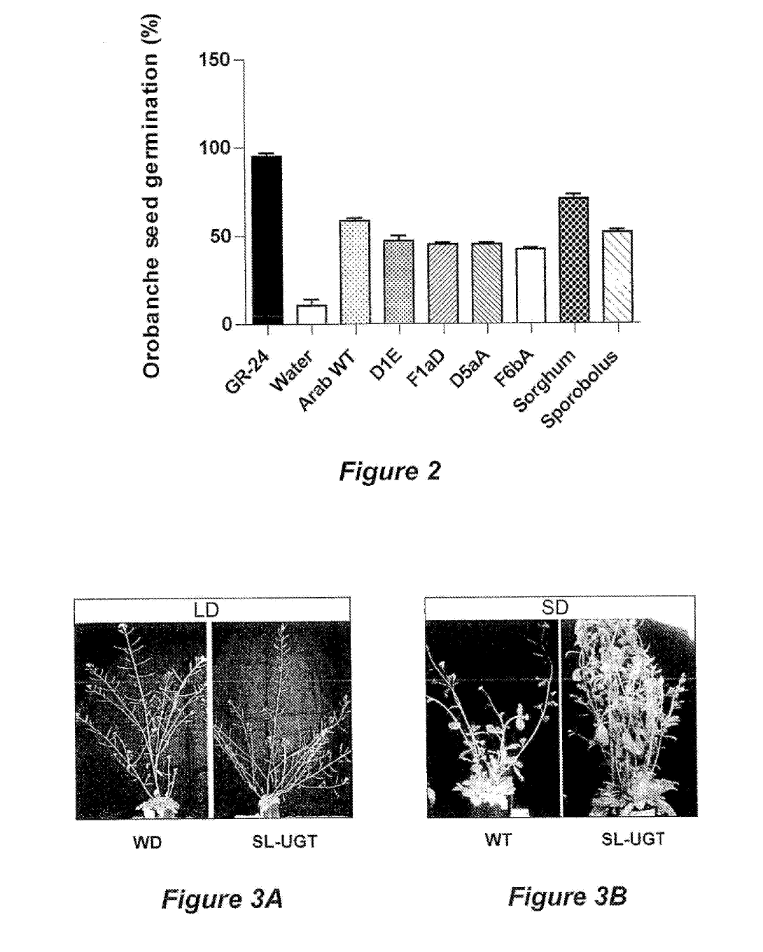

Several Arabidopsis lines expressing SDG8i under the control of the 35S promoter were taken to homozygosity and the levels of the glucosyltransferase product were analysed. The results (not shown) confirm the correlation between transcript levels and protein production in these transgenic lines. To test the ability of SDG8i to glycosylate endogenous strigolactones in vivo, four of these transgenic Arabidopsis lines harbouring the SDG8i gene (designated D1E, F1aD, D5aA and F6bA) were compared with WT Arabidopsis plants for the ability to stimulate Orobanche germination in axenic culture. GR24, sorghum and S. stapfianus were included as controls. The results are shown in FIG. 2; germination of 95% was achieved by treatment with GR24, while the water control induced only 11% germination. The germination percentage of Orobanche by sorghum and S. stapfianus was 71% and 52% respectively, and the 60% germination of Orobanche seeds by WT Arabidopsis plants was significantly higher than that of the all of the transgenic lines tested (p<0.001) with SDG8i transgenic lines stimulating 42-47% germination. Amongst the transgenic lines, F6bA showed the highest reduction in Orobanche germination (30%) compared with the WT Arabidopsis control. These results suggest that SDG8i is able to glycosylate and inactivate at least one of the two or more endogenous strigolactones produced by Arabidopsis.

### Ectopic Expression of SDG8i Affects Shoot Architecture and the Growth Rate

Since it had been found that glycosylation can affect the bioactivity of strigolactones, it was considered that constitutive expression of SDG8i in Arabidopsis would be likely to perturb strigolactone homeostasis and, consequently, provide useful information on the role of strigolactone or strigolactone-like compounds and the connections with interacting response pathways.

Accordingly, the developmental phenotype of seven independent homozygous SDG8i Arabidopsis transgenic lines (designated D1E, F1aD, D5a, D4I, D2E, D7c and F6bA) was examined under long day (LD) and short day (SD) photoperiods. It was found that the SDG8i plants displayed phenotypic differences from those of WT plants that were much more pronounced in SDs (FIG. 3). In particular, it was found that when the plants were grown under LD (ie 16 h day/8 h night), the leaves of four of the SDG8i transgenic lines were significantly longer (about 1.5-1.6 times) compared to WT plants (P<0.05). However, no significant difference was observed in the number of rosette leaves produced before bolting between the SDG8i transgenic and WT plants.

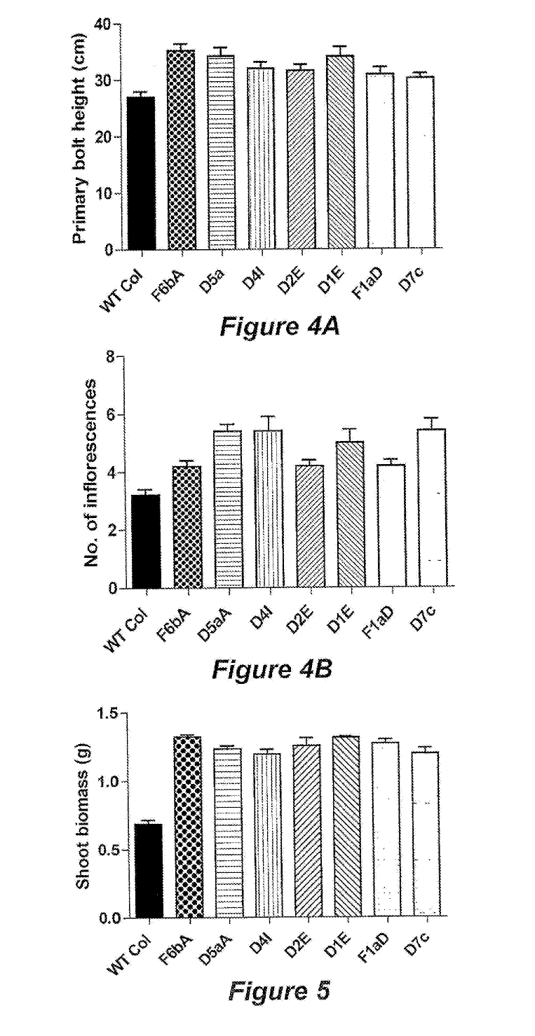

It was also observed that the first inflorescence was produced 16 days after germination, when all of the plants were at about 8 to 10 leaf stage, indicating that the SDG8i gene does not affect flowering time in LD. Moreover, flower morphology and seed development appeared normal in the SDG8i transgenic plants and there was no significant difference between the SDG8i transgenic and WT plants in the size of siliques and number of seeds produced per silique or in the weight of the individual seed produced (data not shown). However, the height of the bolts of all the SDG8i transgenic plants were significantly increased compared to WT plants (P<0.01) after 35 days growth in LD; specifically, the height increase over the WT plants ranged from 15% to 33% (FIG. 4A). Moreover, the SDG8i transgenic plants also produced about 1.3-1.7 times more inflorescences than that of the WT plants (P<0.01), which gave them a bushy appearance (FIG. 4B). In LD, the growth rate of the primary bolt was similar in the SDG8i transgenic and WT plants, with some of the transgenic plants producing a small but significantly higher number of seeds than WT plants (p<0.05) (dry seeds collected following senescence). The larger leaf size and increased number of inflorescences resulted in the average fresh weight of shoot biomass of all of the LD grown SDG8i transgenic plants being almost 1.7-1.8 times higher than that of the WT plants (FIG. 5).

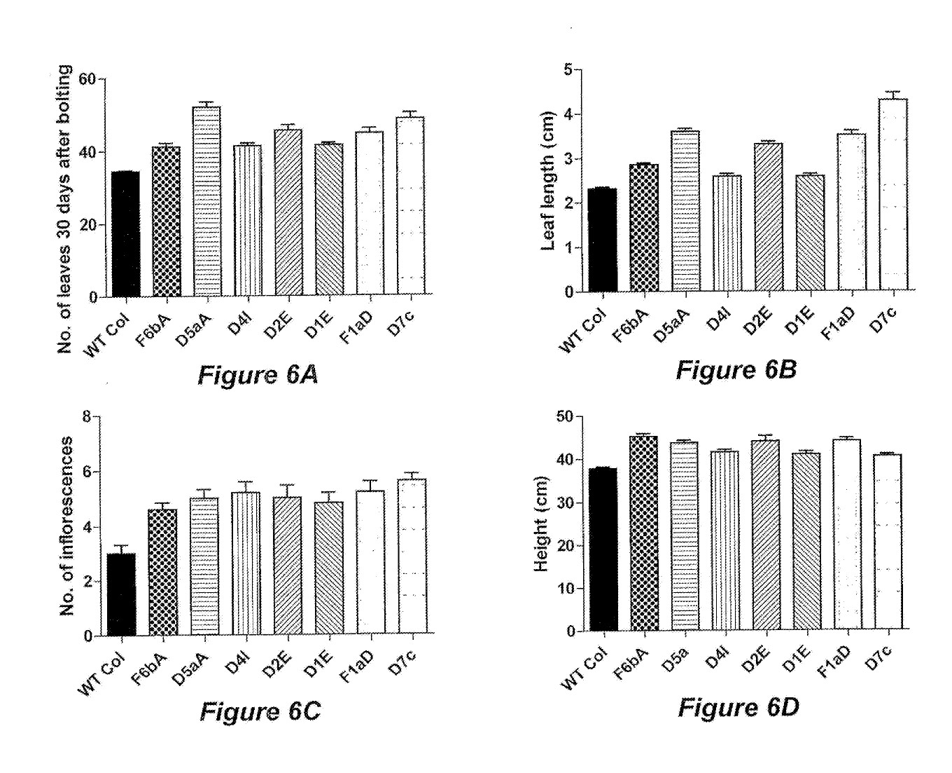

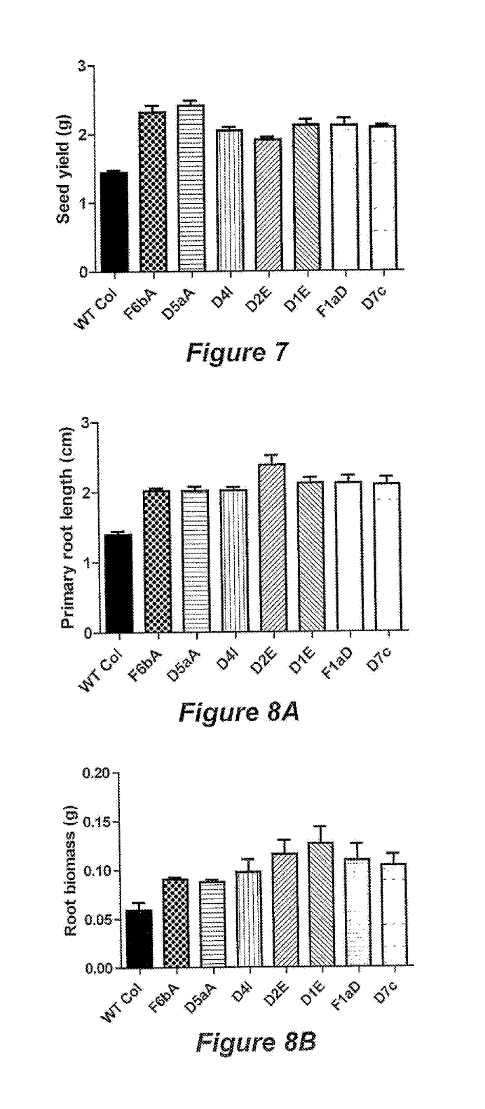

In SD, both SDG8i transgenic and WT plants produced the first inflorescence 52 days after germination. By this time, the WT plants had produced around 30-34 rosette leaves whereas the transgenics had produced approximately 40-42 leaves. Also, a more pronounced difference in the total number of leaves, including primary rosette leaves and leaves produced in the rosette from axillary meristems, formed by SDG8i transgenic and WT plants was observed one month after first flowering. That is, all of the SDG8i transgenic plants produced about 1.3-1.6 times more leaves than the WT plants (P<0.01) (FIG. 6A). In addition, all of the SDG8i transgenic plants produced leaves that were significantly larger than that of the WT plants (P<0.05) (FIG. 6B) and a significantly higher number of inflorescences (ie about 1.4-1.6 times higher) than WT plants (P<0.01) (FIG. 6C). Also, all of the SDG8i transgenic plants were significantly taller than the WT plants after 15 days of bolting (P<0.01) and, after 21 days of bolting, the transgenic lines showed height increases over the WT plants ranging from 9% to 16% (FIG. 6D). Moreover, the rate of growth of the primary inflorescence of transgenic plants was around 20% greater (7-7.12 cm week⁻¹) than that of the WT plants (cf. 5.4 cm week⁻¹). Also, when grown under SD conditions, the increased seed yield of SDG8i transgenic plants previously observed in LD conditions, was found to be more pronounced with the transgenic lines producing about 1.4-1.6 times the amount of seed by mass than the WT plants (p<0.001) (FIG. 7). In both LD and SD, the primary bolt in SDG8i transgenic plants maintained dominance over secondary bolts throughout the growth of the plant (data not shown).

SDG8i Transgenic Plants have Leaves with Increased Cell Size and Number

The rosette leaves of SDG8i transgenic plants are larger than control WT plants when grown in both LD and SD. Following treatment to visualise the cells, the leaves in SD soil-grown plants were examined under the light microscope. It was found that comparable leaves of the transgenic lines contained more cells than the WT plants and the cells in each case were larger in size (see Table 2).

TABLE 2
Leaf area, cell area and total cell number of SDG8i transgenic and WT
Arabidopsis plants

| Plant Name | Leaf Area (mm²) | Cell Area (μm²) | Total Cell Number |
|---|---|---|---|
| WT | 40.50 ± 3.56 | 3160 ± 30 | 12806 ± 1058 |
| D5aA | 57.27 ± 3.41 | 3540 ± 159 | 16325 ± 1666 |
| D41 | 51.93 ± 5.17 | 3575 ± 113 | 14533 ± 1396 |
| D1E | 53.47 ± 4.74 | 3640 ± 65 | 14724 ± 1471 |
| F6bA | 49.57 ± 1.57 | 3592 ± 173 | 13828 ± 359 |

Values shown are the means ± SF of 3 replicates.

The increase in the number and size of leaf cells of the SDG8i transgenic plants indicates that the SL-UGT has a role in promoting leaf cell expansion and cell division.

The Hypocotyl of SDG8i Transgenic Plants is Longer than WT Plants in Dark-Grown Seedlings

Analysis of rice strigolactone synthesis and signalling mutants have indicated that strigolactone negatively regulates cell division, but not cell elongation, in mesocotyls under dark conditions (Hu et al. 2010). To investigate the effect of SL-UGT on SDG8i transgenic plants, seedlings were germinated and grown in the dark and the hypocotyl length measured. It was found that while there was no significant differences in cotyledon development between the dark-grown SDG8i transgenic an WT seedlings, all the transgenic lines produced hypocotyls that were significantly longer, about 1.5-1.7 times, than those of the WT plants (P<0.05)(data not shown).

### SL-UGT Expression Increases Root Cell Length and Lateral Root Initiation

When germinated in the dark, SDG8i transgenic seedlings showed a similar increase in growth of both the primary root and hypocotyl when compared to control WT plants (FIG. 8A). Similarly, when grown in LD conditions, the average fresh weight of the root biomass of all SDG8i transgenic plants was almost twice that of wild-type (FIG. 8B).

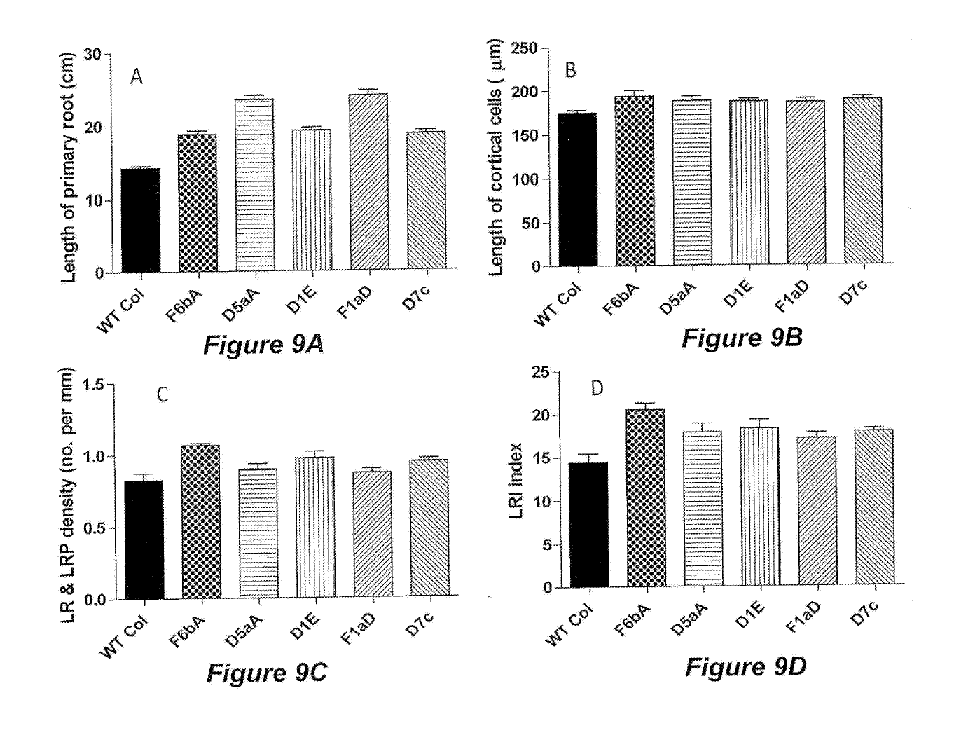

In order to assess whether SL-UGT expression also affected root architecture, a comparison of the root cell length and lateral root primordia in SDG8i transgenic and WT plants grown in vitro in SD was made. In particular, the number of primordia was determined in cleared roots on microscopic slides within the lateral-root-formation zone between the most-distal initiated primordium and the most-distal emerged lateral root (Dubrovsky et al. 2006). Lateral root primordium density (d) was calculated for each individual primary root as number of primordial per mm. I_{LRI}, (the number of lateral root primordia initiated within a portion of the root that corresponds to the length (1, mm) of 100 fully elongated cortical cells in a single file in the same parent root) was determined as 100dl, where l is the average cortical cell length in mm for each individual primary root. As is the case for dark-grown seedlings, the primary root of all of the SDG8i transgenic plants was found to be longer than WT plants grown under SD (p<0.001) (FIG. 9A). Also, the fully elongated cortical cells in the transgenic lines were longer than those of the WT plants (p<0.05), and the calculation of the lateral root primordium density also showed a difference (p<0.005) (FIGS. 9A and B). The higher estimation of I_{LRI} in the SDG8i transgenic plants indicates an increased level of root branching (FIG. 9C). These findings indicate that SL-UGT activity has a positive effect on both primary root growth and lateral root initiation.

### Overexpression of SDG8i Affects Auxin Homeostasis

The spatial distribution of auxin levels control many aspects of plant growth and development including apical dominance, growth of vascular tissue, tropism and organ formation. Strigolactone affects polar auxin transport via its effect on the activity of PINFORMED (PIN) auxin efflux carriers (Leyser, 2005). To examine the effect of constitutive SL-UGT activity on auxin levels and distribution, the SDG8i transgenic lines were crossed with plants containing the DRS-GUS reporter construct (Ulmasov et al., 1997) and the progeny analysed for histochemical GUS activity (Jefferson et al., 1987).

In 4 day-old control WT plants, GUS staining in the hypocotyl was observed along with low level staining in the leaf veins and tips of the cotyledons and root tips. At the same developmental stage, the SL-UGT expressing plants showed essentially the same spatial pattern of GUS activity but with a substantially higher level of staining being observed in the leaf veins and root tips. The difference in staining between the SDG8i transgenic and WT plants was more pronounced at the two-leaf stage with the transgenic lines showing much more GUS activity in the vascular tissue of shoots and roots and high level GUS activity at the leaf margins. These results indicate that endogenous auxin levels are elevated in SDG8i plants.

### SDG8i Transgenic Plants Exhibit Salt and Freezing Tolerance

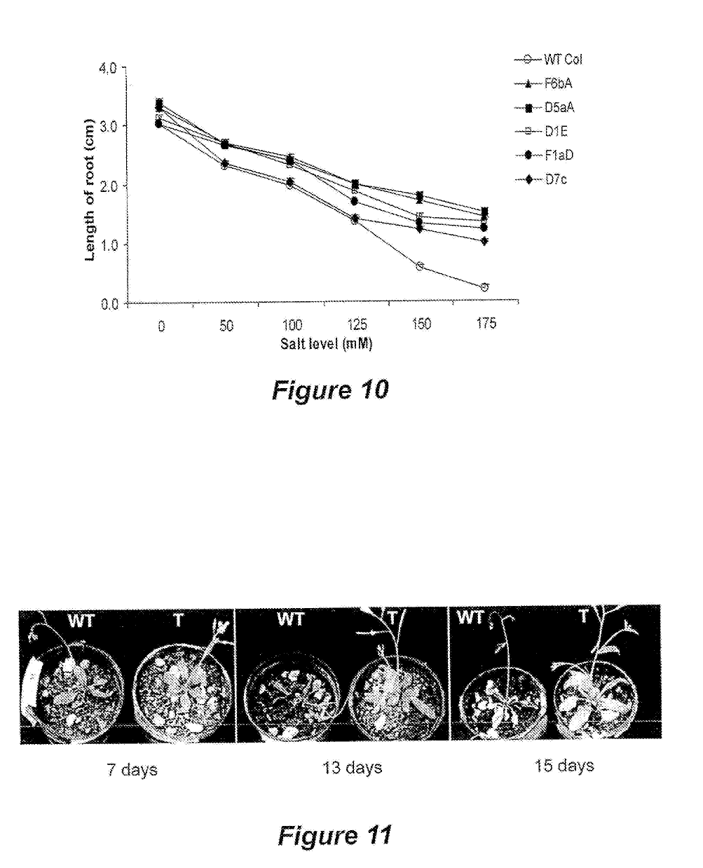

A comparison of the response of SDG8i transgenic and WT plants to growth on high salt media was made. The results are shown in FIG. 10. No significant difference in primary root growth was observed between the SDG8i transgenic and WT seedlings after 7 days growth at salt concentrations below 100 mM. However, at 150 mM NaCl, all of the transgenic lines showed significantly greater salt resistance with 2-3 times more root growth than that of the WT plants (p<0.001).

Wild-type plants grown in 175 mM NaCl showed severe inhibition of root growth. At this salt level, shoot growth was inhibited less than root growth, possibly because the rate of transpiration in the plates was too low to cause a build-up of high levels of NaCl in the shoot. In contrast, at 175 mM NaCl, the root growth of the transgenic lines was 5-7 times that of the WT plants (p<0.001). At 200 mM NaCl, the root growth of the SDG8i transgenic plants was almost completely inhibited.

A freezing tolerance test was also performed on the SDG8i transgenic and WT control plants. It was found that all plants exposed to −4° C. freezing stress survived. However, at −8° C., all of the transgenic lines survived, whereas the survival rate of the WT plants was 83%. Moreover, at −12° C., the control WT plants were affected greatly with a survival rate of only 34%, but most of the transgenic lines survived freezing at this temperature with a survival rate ranging from 69% to 72% (Table 3).

TABLE 3
Percent survival of SDG8i transgenic and WT Arabidopsis
plants in freezing tolerance test

| | Number of plants | Percentage of plant |
|---|---|---|
| Plant Name | surviving at −12° C. | survival at −12° C. |
| WT | 16.33 ± 0.88 | 34 |
| F6bA | 36 ± 0.58 | 72 |
| D5aA | 35 ± 1.15 | 70 |
| D1E | 36 ± 1.53 | 72 |
| F1aD | 34.33 ± 0.33 | 69 |

Values are the means ± SE of 3 replicates.

### SDG8i Transgenic Plants are Drought Tolerant

After 7 days without watering (waterholding treatment), the first signs of wilting was visible in WT plants, while all SDG8i transgenic plants retained a healthy appearance (FIG. 11a). After 13 days of drought, all WT plants suffered clearly from water loss and were severely dehydrated; however, all SDG8i transgenic plants still appeared healthy (FIG. 11b). On day 14 of waterholding treatment, all plants were rewatered to allow recovery. On day 15, 24 hours after rewatering, all SDG8i transgenic plants had regained turgor, while none of the WT plants showed any sign of recovery (FIG. 11c). To quantify the increase in drought tolerance conferred by the expression of SL-UGT in the SDG8i transgenic lines, a measure of protoplasmic drought tolerance (PDT; defined as the percent relative humidity at which 50% of leaf cells survive) was obtained.

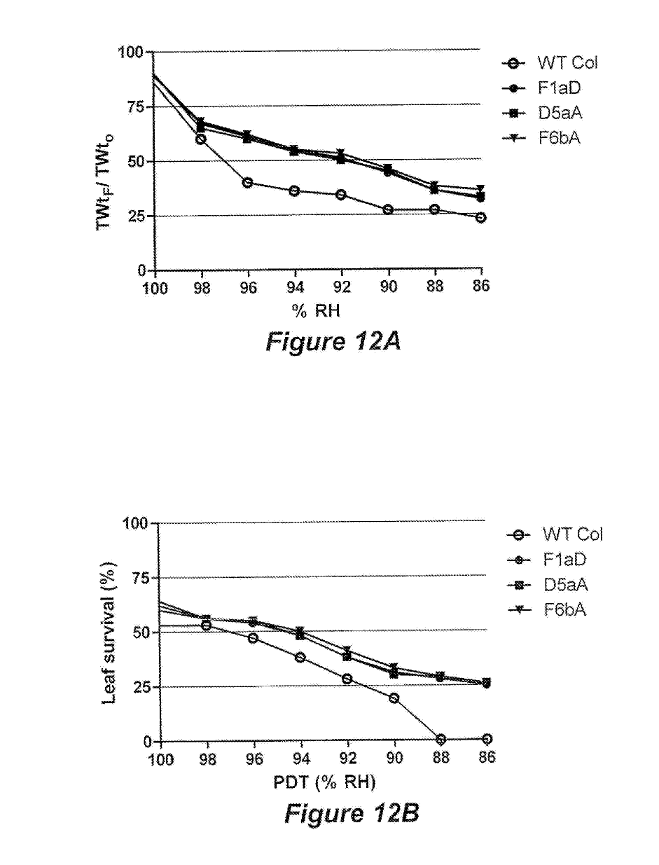

The PDT of WT plants occurred at 97% RH whereas the PDT of the transgenic lines F1aD, D5aA and F6bA did not occur until RH reached 92.5%, 92% and 91% respectively (FIG. 12A). The difference of 5-6% RH units indicates that SL-UGT activity mediates a substantial improvement in PDT. Moreover, when the ratio of TWt_{F}/DWt_{F}/TWtₒ/DWtₒ was used as an index of damage, the transgenic lines showed no injury above 88% RH, compared to the WT plants which showed injury below 96% RH. Subjective estimates of the % of live leaves for each plant also showed a significant difference between the SDG8i transgenic and WT plants. In particular, it was found that all of the leaves of WT plants were discoloured and dead at 88% RH, whereas the transgenic lines retained around 27% healthy green leaves at 86% RH (FIG. 12B). Further, using a neutral red uptake test on hand sections of live leaves from SDG8i transgenic and WT plants at 90% RH, showed that a substantially higher number of damaged cells are present in the control WT plants compared to the transgenic lines (data not shown).

### Discussion

It has been shown that SL-UGT activity confers a large number of desirable traits in non-resurrection plants without any adverse effects on plant development or morphology. In particular, it has been found that the SDG8i transgenic Arabidopsis plants produced in this example form twice the leaf mass and flowering bolts within the same time period, when compared to WT plants. This results in a 65% increase in seed yield. In addition, the SDG8i transgenic plants have substantially increased drought-tolerance as well as exhibiting enhanced salt and cold tolerance. Moreover, the transgenic lines show enhanced resistance to attack from parasitic plants (Orobanche and Striga) which can devastate yields (around 80% losses) in many monocot and dicot crop species leading to annual crop losses world-wide that have been estimated to be in the tens of billions of dollars (Tsuchiya and McCourt, 2011).

While not wishing to be bound by theory, it is considered that a reduced level of free strigolactone and/or strigolactone-like compounds and/or increases in glycosylated strigolactone and/or strigolactone-like compounds resulting from the expression of the SL-UGT drives the phenotypic changes observed in the SDG8i transgenic plants. The pivotal role that phytohormones play in regulating plant growth and stress resistance underpins the approach of manipulation of phytohormone biosynthesis and signalling as a means of genetically engineering desired traits into crop plants. Perhaps the best known example is the substantial increases in wheat yields that were obtained during the “green revolution” of the 1960s and 70s by selecting plants containing genes that decreased sensitivity to the phytohormone, gibberellin. It was proposed then that the increased yield of these “green revolution” plants resulted from a redirection of plant resources from vegetative biomass into increased seed production (Hedden, 2003). This concept of redirection of limited resources has been reinforced by manipulations to enhance resistance to biotic and abiotic stresses, which, in many cases, have produced plants of reduced stature. The SDG8i transgenic plants produced in this example however, have demonstrated that an increase in seed yield, vegetative biomass and stress resistance can occur concomitantly and supports the notion that reduced/redirected growth has evolved as an active part of the overall survival strategy of the plant rather than being directly related to resource-limitations.

### Example 2

### Identification of Orthologues of SDG8i

### Materials and Methods

Searches were conducted in the non-redundant GenBank CDS database and Arabidopsis database (The Arabidopsis Information Resource (TAIR) database) using NCBI blastp 2.2.27 search with amino acid sequence from the SL-UGT encoded by SDG8i. Since it is believed that the substrate specificity of UGTs is conferred by amino acid sequence in the N-terminal region of the proteins, the search in the above databases was undertaken using the amino acid sequence from amino acid 1 to 340 of the sequence shown as SEQ ID NO: 3. This also excluded the PSPG region (amino acid 345 to 390 of the sequence shown as SEQ ID NO: 3) which is conserved in all UGTs.

### Results

The searches identified orthologue sequences present in barley (H. vulgare subsp. vulgare) and Arabidopsis (Accession No AT3G16520.3), with amino acid sequence identities (within the N-terminal regions) of 66% and 38%, respectively. When compared using the full-length amino acid sequences, the sequence identity between the SL-UGT encoded by SDG8i and the barley protein was 67%. The sequence identity between the full-length SL-UGT encoded by SDG8i and the Arabidopsis protein was 41%.

### Discussion

Orthologues of SDG8i were found in barley and Arabidopsis that are expected to be suitable for use in the present invention. Using the nucleotide sequence for the coding region of SDG8i, additional related proteins were identified encoded by Arabidopsis sequence Accession No AT1G1390.1 (53.6% S nucleotide sequence identity) and Phyllostachys edulis (bamboo) cDNA clone bphylf043fl9 (Accession No emb FP091958.1) (78% nucleotide sequence identity). These nucleotide sequences may also be suitable for use in the present invention.

### Example 3

### Prophetic Example of the Introduction of SDG8i into B. napus

Brassica napus transformants expressing the SDG8i gene under the control of the CaMV 35S promoter in the vector pMDC32 (Curtis et al., 2003) will be obtained using an A. tumefaciens floral dip method (Verma et al., 2008). Flowering B. napus plants will be submerged in an Agrobacterium culture harbouring the SDG8i construct and the resulting seeds grown under selection for resistance to hygromycin. Hygromycin resistant plants will be taken through to the second generation (T2) to generate plants homozygous for SDG8i. Expression of the gene will be confirmed using RT-PCR. Transformants will then be characterised. It is expected that these plants will exhibit enhanced growth characteristics, increased seed yield, increased stress tolerance and reduced susceptibility to the parasitic plant Orobanche.

### Example 4

### Activation of SDG8i by the Transcription Factors SDG10y and SDG7y

Use of the regulatory region of the SL-UGT gene, SDG8i, as bait in the yeast one-hybrid system resulted in the isolation of two transcription factor (TF) genes, SDG10y and SDG7y from S. stapfianus. Both SDG10y and SDG7y encode members of the AP2 (APETALA2)/ERF (Ethylene Response Factor) transcription factor super family. Both SDG10y and SDG7y are detectable in fully hydrated plants, but have been found to decrease to below detectable levels around 50-40% relative water content (RWC) (Le, 2004). Subsequent analysis indicated that these transcription factors and, particularly SDG10y, are positive activators of SDG8i. It is considered that the SDG10y and SDG7y transcription factors are activated post-translationally by water-loss and bring about the activation of genetic components of the desiccation tolerance program (including the SDG8i gene). This example aimed to examine the viability of introducing into a plant, the SDG10y or SDG7y gene to increase the transcription of an SL-UGT gene such as SDG8i.

### Materials and Methods

Activation of GUS Expression Driven by prSDG8i

The promoter sequence (1660 nt) of SDG8i was placed upstream of the GUS reporter gene in a construct designated prSDG8i-GUS. This construct was transiently co-transformed into onion epidermal peels along with the pMDC32:7y or pMDC32:10y constructs which contained either SDG10y or SDG7y under the transcriptional regulation of the CaMV 35S promoter which is constitutively active in onion cells. The formation of a blue precipitate indicates activation of the SDG8i promoter-driven GUS reporter gene by the SDG10y or SDG7y transcription factor constructs.

Agrobacterium-mediated transient transformation was achieved by immersing onion peels in the Agrobacterium suspensions containing these constructs in 10 mM MES buffer (pH 5.5) and 200 μM acetosyringone at 28° C. in the dark for 2/3 days. A 35S:GUS control construct was included in these experiments and the hormone methyl jasmonate (MJ) was also added to some suspensions. After 2 days, the onion peels were soaked in a GUS staining solution and left at 28° C. in the dark. The onion peels were examined every 2 hours to monitor the formation of blue precipitate.

### Generation of Transgenic Plants Expressing SDG10y and SDG7y

Transgenic lines expressing SDG10y and SDG7y under the control of the CaMV 35S promoter were produced in Arabidopsis.

The function of the transcription factors was examined by looking at their effects on plant development and stress responses, such as cold, salt and drought, firstly in Arabidopsis, Six independent homozygous SDG10y and three SDG7y Arabidopsis transgenic lines were produced.

The transgenic plant lines were analysed in a manner similar to that described above for SDG8i transgenic plants in Example 1.

### Results

SDG10y and SDG7y Transcription Factors Increase prSDG8i-Driven GUS Expression

GUS activity was observed in onion peels containing 35S:GUS control constructs, indicating that GUS activity is detectable within this transient system. Onion peels containing the prSDG8i-GUS construct by itself also showed a small amount of GUS activity which was elevated in the presence of MJ. SDG8i has previously been shown to be an MJ-responsive gene (Le, 2004).

In contrast to the above results, when there was co-expression of SDG10y, there was a substantial increase in expression of the SDG8i promoter-driven GUS reporter. Similar results were obtained with SDG7y, although the results did indicate that the SDG7y transcription factor is less effective than SDG10y at activating SDG8i expression under these experimental conditions.

### Transgenic Lines Similar to SDG8i Transgenic Plants

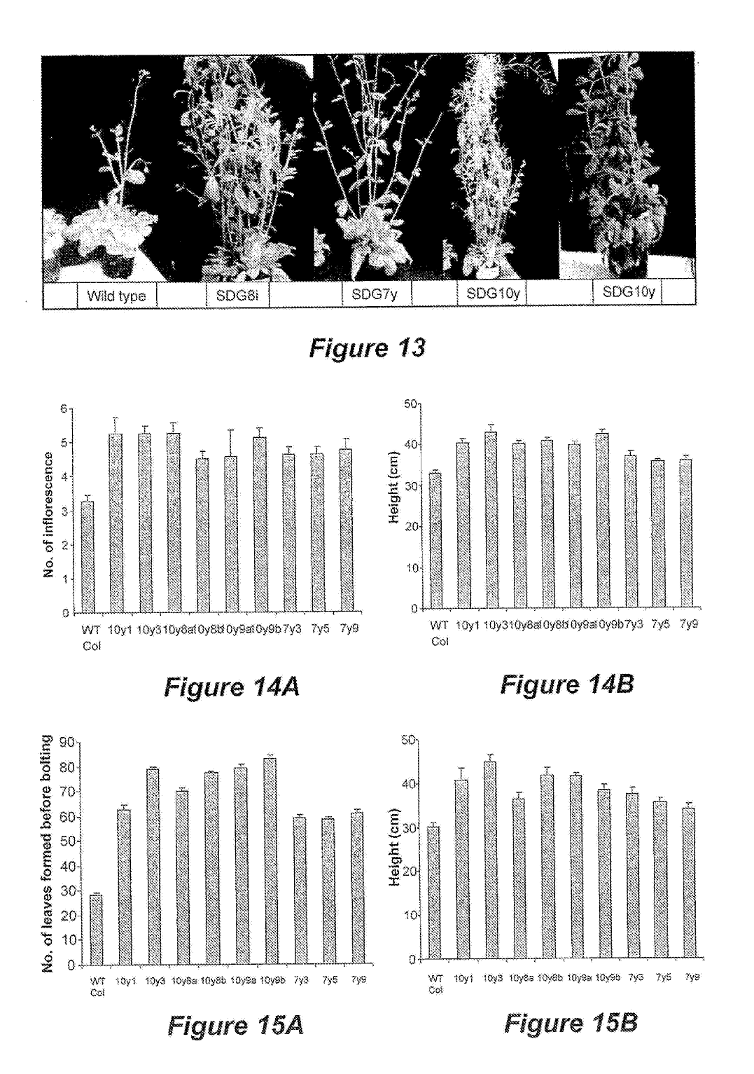

When the developmental phenotype of the SDG10y and SDG7y homozygous Arabidopsis transgenic plant lines were examined under different day length periods they exhibited very similar characteristics to those of the SDG8i transgenic plants (see FIG. 13). In long day (LD) period (16 h day/8 h night), both the SDG10y and SDG7y transgenic lines were taller (P<0.05) compared to wild-type (WT) plants and produced a significantly higher number of inflorescences (P<0.01), compared to WT plants (FIGS. 14A and 14B), which gave them the same bushy appearance as SDG8i transgenic plants.

As is also the case for SDG8i transgenic plants, when grown in short day (SD) length (8 h day/16 h night), the differences to WT control plants were more pronounced with the number of rosette leaves in the transgenic plants during bolting being 2 to 3 times higher compared to that of WT plants. SDG10y transgenic plants produced a higher number of rosette leaves compared to that of SDG7y transgenic plants (FIG. 15A). The leaf size of all of the transgenic plants was almost twice the size of that of WT plants. In addition, the height of the SDG10y and SDG7y transgenic plants measured after 40 days of bolting (FIG. 15B), was almost 1.3 to 1.5 times higher than that of the WT plants; all SDG10y transgenic plants being significantly taller than the SDG7y transgenic plants. Further, the rate of growth for transgenic plants was about 6.7 to 7.5 cm week⁻¹, whereas the growth rate for WT plants was 5.7 cm week⁻¹ which was about 76% to 87% of that of transgenic plants. The flowers of all transgenic lines and WT plants were normal, fertile and similar in size. The size of siliques and number of seeds produced per silique was also similar in all transgenic lines and WT plants.

Moreover, preliminary experiments have indicated that the SDG10y and SDG7y transgenic plants also show stress resistance.

### Discussion

It has been shown that an alternative approach to the introduction of an SL-UGT-encoding sequence into plants, is the introduction of a sequence encoding a relevant transcription factor such as SDG10y and SDG7y (so as to increase transcription of an endogenous gene encoding an SL-UGT). The similarity in the phenotypic development of SDG10y and SDG7y transgenic plants produced in this example to that of the SDG8i transgenic plants of Example 1 suggests that the SDG10y and SDG7y transcription factors activate the Arabidopsis SL-UGT orthologue to the S. stapfianus SDG8i gene.

### Example 5

### Prophetic Example of the Introduction of SDG8i into Plant Species Using Transbacter Technology

The SDG8i coding region described in Example 1 can be transgenically introduced into a number of different plant species (including Arabidopsis, canola and rice species) using the Transbacter system (Cambia, Canberra, Australia). The transbacter system utilises a number of bacterial species such as Rhizobium sp. NGR234, Sinorhizobium meliloti and/or Mesorhizobium loti, which are made competent for gene transfer by the acquisition of both a disarmed Ti plasmid and a binary vector such as pCAMBIA vectors pC1105.1 and pC1105.1r (see Broothaerts et al., 2005, the entire disclosure of which is hereby incorporated by reference, including the associated Supplementary data; Chilton 2005); or by the acquisition of a unitary vectors such as pCAMBIA vectors pCAMBIA5106 or pCAMBIA 5105.

The transbacter system utilises any of the following combination of bacterial strains and plasmids:

### TransBacter Strains/Unitary Vectors

Rhizobium leguminosarum bv. Trifolii strain ANUS45+pCAMBIA5106 (Km and Spec selection)
Sinorhizobium meliloti+pCAMBIA 5105 (Km and Spec selection)

### TransBacter Strains/Binary Vectors

Mesorhizobium loti+pWBTi1+pCAMBIA1105.1R (Km and Spec selection)
Rhizobium sp. NGR 234+pWBTi1+pCAMBIA1105.1R (Km and Spec selection)
Rhizobium sp. NGR 234+pWBTi3+pCAMBIA 1105.1R (Km and Spec selection)
Sinorhizobium meliloti+pWBTi1+pCAMBIA1105.1R (Km and Spec selection)
Sinorhizobium meliloti+pWBTi3 (Km selection)
Sinorhizobium meliloti+pWBTi3+pCAMBIA1105.1R (Km and Spec selection)

Plasmid maps of the above mentioned vectors are published in Broothaerts et al., 2005 and/or on the Cambia website.

Transformation can be performed using standard protocols, for example, using minor variations of standard leaf disk, scutellum-derived callus or floral dip transformation methods (see Broothaerts et al., 2005). The resulting transgenic plants will express hygromycin resistance and GUS activity, the number of copies of T-DNA will be examined by Southern blot analysis, T-DNA insertions into the host genome will be examined by PCR-mediated sequencing of integration sites (see Broothaerts et al., 2005).

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

All publications mentioned in this specification are herein incorporated by reference. Any discussion of documents, acts, materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of each claim of this application.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

Abdul R et al., Triticeae Genomics and Genetics 1 (2) (2010).
Arencibia A et al., Biotechnologia Aplicada 9 (2):156-165 (1992).
Augusti et al., Proc Natl Acad Sci USA 108:20242-20247 (2011).
Benfey et al. EMBO J 8:2195-2202 (1989).
Benfey and Chua Science 250:959-966 (1990).
Blomstedt C K et al., Plant Growth Regul 62:217-232 (2010).
Broothaerts et al., Nature 433: 629-33 (2005).
Casas A M et al., Proc Natl Acad Sci USA 90:11212-11216 (1993).
Chabaud m et al., Plant J 7:253-260 (1995).
Chilton Nature Biotechnol 23 (3): 309-310 (2005)
Christou P et al., Handbook of Plant Biotechnology, John Wiley and Sons (2004).
Cousins Y L et al., Aust J Plant Physiology 18:481-494 (1991).
Curtis M D and U Grossniklaus, Plant Physiol 133:462-469 (2003).
da Silva JAT (ed), Floriculture, Ornamental and Plant Biotechnology Volume 2, Global Science Books.
Dun E A et al., Trends in Plant Science 14:364-372 (2009).
Dubrovsky et al., Annals of Botany 103: 807-817 (2009).
El-Awady M et al., Arab J Biotech 11 (1):71-84 (2008).
Gaff D F and D J Carr, Australian Journal of Biological Sciences 14:299-301 (1961).
Gaff D F et al., Funct Plant Biol 36:1-11 (2009).
Hedden P, Trends in Genetics 19:5-9 (2003).
Ho N H et al., Omonrice 9:30-35 (2001).
Hruz T et al., Advances in Bioinformatics Article ID 420747, 5 pages (2008).
Hu Z et al., Plant Cell Physiol 51:1136-1142 (2010).
Jackson R G et al., J Biol Chem 276:4350-4356 (2001).
Jefferson R A, Plant Mol Biol Rep 5:387-405 (1987).
Kapulnik Y et al., Planta 233:209-216 (2011).
Kohlen W et al., Plant Physiology 155:974-987 (2011).
Koltai H. New Phytologist doi: 10.1111/j. 1469-8137.2011.03678.x (2011).
Le T-N. Genetics of dessication tolerant plants in resurrection plant Sporobolus stapfianus. PhD thesis, Monash University, Melbourne VIC, Australia. Le et al., Functional Plant Biology 34:589-600 (2007).
Leyser O, Current Opinion in Genetics and Development 15:468-471 (2005).
Leyser O, Plant, Cell & Environment 32:694-703 (2009).
Li F et al., J Plant Physiol 145:87-97 (2007).
Lim E-K et al., EMBO J. 23:2915-2922 (2004).
López-Ráez J A et al., New Phytologist 187:343-354 (2010).
Luschnig et al., Genes Dev 12:2175-2187 (1998).
Marillonnet S et al., Nature Biotech 23:718-723 (2005).
Martinez-Trujillo M, et al., Plant Mol Bio. Rep 22:63-70 (2004).
Mizoi J et al., Biochim Biophys Acta 1819:86-96 (2012).
Neale A D et al., Plant Cell & Environment 23:265-277 (2000).
Ouwerkerk P B and A H Meijer, Curr Protoc Mol Biol Chpt 12:Unit 12.12 (2001).
Owen P C. J Exp Bot 3:118 (1952).
Rech E L et al., Nat Protoc 3 (3):410-418 (2008).
Ruyter-Spira C et al., Plant Physiology 155:721-734 (2011).
Samac and Shah, Plant Cell 3:1063-1072 (1991).
Sambrook J et al., Molecular Cloning: A Laboratory Manual, 2nd Ed., Cold Spring Harbor Laboratory, Cold Spring Harbor, N.Y. (1989).
Scott M P., Transgenic maize: methods and protocols, SpringerHumana Press (2009).
Slater A et al., Plant Biotechnology—The Genetic manipulation of Plants, Oxford University Press (2003).
Slatyer R O. Australian Journal of Agricultural Research 6: 365-377 (1955)
Snowden K C et al., The Plant Cell 17:746-759 (2005).
Stepanova A N et al., Current Opinion in Plant Biology 12:548-555 (2009).
Tsuchiya Y et al., Mol Biosyst 8:464-469 (2012).
Ulmasov T et al., The Plant Cell 9:1963-1971 (1997).
Umehara M et al., Plant and Cell Physiology 51:1118-1126 (2010).
Verma S S et al., J Plant Biochemistry & Biotechnology 17:197-200 (2008).
Wang Z-Y and GE Yaxin. In vitro Cell Dev Biol.—Plant 42:1-18 (2006).
Woo H R et al., The Plant Cell 13:1779-1790 (2001).
Yoneyama K et al., Planta 227:125-132 (2007).

1. A plant cell of, or derived from, a plant comprising an exogenous polynucleotide molecule encoding a uridine diphosphate glucose (UDP) glycosyltransferase (UGT) and/or an exogenous polynucleotide molecule which increases transcription of an endogenous gene encoding a UGT, wherein said UGT is a UGT which glycosylates one or more strigolactone or strigolactone-like compound (ie an SL-UGT).

2. The plant cell of claim 1, wherein the plant cell is selected from crop and forage species of plants, and hybrids thereof.

3. The plant cell of claim 2, wherein the plant cell is of the species Brassica napus (canola).

4. The plant cell of claim 1, wherein the plant cell is selected from species and hybrids of lawn grasses and ornamental plants.

5. The plant cell of claim 1, which forms part of a multicellular structure.

6. The plant cell of claim 1, wherein the exogenous polynucleotide molecule encodes a SL-UGT.

7. The plant cell of claim 6, wherein the exogenous polynucleotide molecule encodes a SL-UGT comprising an amino acid sequence that has at least 35% sequence identity to the amino acid sequence shown as SEQ ID NO: 3 or a biologically active fragment thereof.

8. The plant cell of claim 6, wherein the exogenous polynucleotide molecule encodes a SL-UGT comprising an amino acid sequence that has at least 98% sequence identity to the amino acid sequence shown as SEQ ID NO: 3 or a biologically active fragment thereof.

9. The plant cell of claim 6, wherein the exogenous polynucleotide molecule encodes a SL-UGT comprising an amino acid sequence that is identical or substantially identical to the sequence shown as SEQ ID NO: 3 or a biologically active fragment thereof.

10. The plant cell of claim 1, wherein the exogenous polynucleotide molecule encodes a transcription factor that increases transcription of an endogenous gene encoding a UGT.

11. The plant cell of claim 10, wherein the exogenous polynucleotide molecule encodes a transcription factor comprising an amino acid sequence that has at least 35% sequence identity to the amino acid sequence shown as SEQ ID NO: 6 (or a biologically active fragment thereof) or SEQ ID NO: 7 (or a biologically active fragment thereof).

12. The plant cell of claim 10, wherein the exogenous polynucleotide molecule encodes a transcription factor comprising an amino acid sequence that has at least 98% sequence identity to the amino acid sequence shown as SEQ ID NO: 6 (or a biologically active fragment thereof) or SEQ ID NO: 7 (or a biologically active fragment thereof).

13.-15. (canceled)

16. A method of producing a plant with one or more improved or introduced desirable traits, said trait(s) being selected from the group consisting of growth rate, cold tolerance, salt tolerance, drought tolerance, and resistance to parasitic plants, said method comprising introducing to said plant an exogenous polynucleotide molecule encoding a uridine diphosphate glucose (UDP) glycosyltransferase (UGT) and/or an exogenous polynucleotide molecule which increases transcription of an endogenous gene encoding a UGT, wherein said UGT is a UGT which glycosylates one or more strigolactone or strigolactone-like compound (ie an SL-UGT).

17. The method of claim 16, wherein the plant is selected from crop and forage species of plants, and hybrids thereof, and species and hybrids of lawn grasses and ornamental plants.

18. The method of claim 17, wherein the plant is of the species Brassica napus (canola).

19. The method of claim 16, wherein the exogenous polynucleotide molecule encodes a SL-UGT.

20. The method of claim 19, wherein the exogenous polynucleotide molecule encodes a SL-UGT comprising an amino acid sequence that has at least 35% sequence identity to the amino acid sequence shown as SEQ ID NO: 3 or a biologically active fragment thereof.

21. The method of claim 19, wherein the exogenous polynucleotide molecule encodes a SL-UGT comprising an amino acid sequence that has at least 98% sequence identity to the amino acid sequence shown as SEQ ID NO: 3 or a biologically active fragment thereof.

22. The method of claim 19, wherein the exogenous polynucleotide molecule encodes a SL-UGT comprising an amino acid sequence that is identical or substantially identical to the sequence shown as SEQ ID NO: 3 or a biologically active fragment thereof.

23. The method of claim 16, wherein the exogenous polynucleotide molecule encodes a transcription factor that increases transcription of an endogenous gene encoding a UGT.

24.-26. (canceled)
